(19) United States
(12) Patent Application Publication
(10) Pub. No.: US 2014/0057251 A1
(43) Pub. Date: Feb. 27, 2014

(54) Cannabis Genomes and Uses Thereof

(71) Applicant: Kevin McKernan, Marblehead, MA (US)

(72) Inventor: Kevin McKernan, Marblehead, MA (US)

(73) Assignee: Medicinal Genomics Corporation, Woburn, MA (US)

(21) Appl. No.: 13/588,935

(22) Filed: Aug. 17, 2012

Related U.S. Application Data
(60) Provisional application No. 61/600,436, filed on Feb. 17, 2012.
(60) Provisional application No. 61/575,329, filed on Aug. 18, 2011.

(51) Int. Cl. C07K 16/40; C12Q 1/68; G01N 33/573; C12N 9/88

(52) U.S. Cl. CPC C07K 16/40; C12N 9/88; C12Q 1/68; G01N 33/573
USPC 435/6.11; 536/23.2; 530/387.1; 435/232; 435/320.1; 435/419; 435/7.4

(57) ABSTRACT

Using the efficiency of next generation sequencing, a draft de novo reference sequence for the Cannabis (C.) Sativa and C. Indica genomes has been generated as well as four full length contiguous sequences with homology to THCA and CBDA synthases and 10 partially homologous contigs with truncated ORFs. In particular aspects the invention is directed to an (one or more) isolated sequence (e.g., nucleic acid sequence, DNA, RNA, genomic sequence, polypeptide) of a Cannabis genome and uses thereof.

### RELATED APPLICATIONS

This application claims the benefit of U.S. Provisional Application No.61/600,436, filed on Feb. 17, 2012, and U.S. Provisional Application No. 61/575,329 filed on Aug. 18, 2011. The entire teachings of the above applications are incorporated herein by reference.

### INCORPORATION BY REFERENCE OF MATERIAL IN ASCII TEXT FILE

This application contains sequences (SEQ ID NO: 1 and SEQ ID NO: 2) and information concerning the sequences (annotated genome and single nucleotide polymorphisms) that are contained on two duplicate copies (Copy 1 and Copy 2) of four (4) compact disks all of which are herein incorporated by reference. The contents of Copy 1 and Copy 2 for each of the three compact disks are identical.

Each disk, which was filed in U.S. Provisional Application No. 61/575,329 on Aug. 18, 2011 and to which a benefit of priority is claimed herein, is identified as follows:

### Disk 1 of 4

Copy 1 and Copy 2 of Disk 1 of 4 each contain the following:
File name:
App-Indica_SEQ_ID_No2.txt; created Aug. 18, 2011; 332,049 KB in size

### Disk 2 of 4

Copy 1 and Copy 2 of Disk 2 of 4 each contain the following:
File name:
App-Sativa_SEQ_ID_No1.txt, created Aug. 18, 2011; 298,667 KB in size
GenomeAnnotationForSativaGenome.txt created Aug. 18, 2011; 16,367 KB in size.

### Disk 3 of 4

Copy 1 and Copy 2 of Disk 3 of 4 each contain the following:
File name:
Mapped_{—}Sativa_to_{—}Indica_for_SNP.txt; created Aug. 18, 2011; 418,535 KB in size.

### Disk 4 of 4

Copy 1 and Copy 2 of Disk 4 of 4 each contain the following:
File name: App-Sativa_SEQ_ID_No1 PDF 1 of 2.pdf; created Aug. 18, 2011; 95,742 KB in size.
File name: App-Sativa_SEQ_ID_No1 PDF 2 of 2.pdf; created Aug. 18, 2011; 88,621 KB in size.
File name: App-Indica_SEQ_ID_No2 PDF 1 of 2.pdf; created Aug. 18, 2011; 82,952 KB in size.
File name: App-Indica_SEQ_ID_No2 PDF 2 of 2.pdf; created Aug. 18, 2011; 102,174 KB in size.

### BACKGROUND OF THE INVENTION

The non-psychoactive cannabinoid, cannabidiol has recently been shown to promote apoptosis in tumor cells. Eighty four (84) other cannabinoids have been measured in Cannabis sativa but the genetics governing the synthesis of all of these compounds are only partially known.

### SUMMARY OF THE INVENTION

Described herein is a de novo assembly of the medicinal plants Cannabis Sativa and Cannabis Indica. These diploid assemblies range in size from 280 Mb to 303 Mb, are 67% AT, and have mitochondrial genomes up to 366 Kb. Of particular interest is a mPIF transposon mediated copy number variation in the synthase genes responsible for cannabigerol acid (CBGA) conversion to tetrahydrocannabinol (THC). Also evident is high diversity in the limonene and alpha pinene synthases. In total, the data provided herein increases the available knowledge on the sequence on this plant over 70,000 fold and over 98.6% of the Cannabis sequence in Genbank has been covered with the 300 Mb assemblies described herein. These data provide selective breeding strategies to maximize medicinal expression and attenuate psychoactive content while also providing a tool for genetic prediction of cannabinoid expression and chemotypes at seedling stages.

Accordingly, in one aspect, the invention is directed to a nucleic acid comprising a nucleotide sequence that has about 82% to SEQ ID NO: 3, SEQ ID NO: 5, SEQ ID NO: 7 or SEQ ID NO: 9 or a portion thereof that encodes a biologically active cannabinoid synthase, or a complement thereof. In aparticular aspect, the invention is directed to nucleic acid comprising SEQ ID NO: 3, SEQ ID NO: 5, SEQ ID NO: 7 or SEQ ID NO: 9 or a portion thereof that encodes a biologically active cannabinoid synthase, or a complement thereof.

In another aspect, the invention is directed to a polypeptide comprising an amino acid sequence that has about 67% identity to SEQ ID NO: 4, SEQ ID NO: 6, SEQ ID NO: 8 or SEQ ID NO: 10 or a biologically active portion thereof, such as a biologically active portion that functions as a cannabinoid synthase. In a particular aspect, the invention is directed to a polypeptide comprising SEQ ID NO: 4, SEQ ID NO: 6, SEQ ID NO: 8 or SEQ ID NO: 10 or a biologically active portion thereof, such as a biologically active portion that functions as a cannabinoid synthase.

Other aspects of the invention include an antibody that specifically binds one or more polypeptides described herein. Also encompasses by the inventions are vectors comprising the nucleic acid sequences provided herein and cells comprising the vectors.

In another aspect, the invention is directed to a method of producing a Cannabinoid synthase comprising maintaining a cell comprising a vector comprising the nucleic acid sequences provided herein under conditions in which the Cannabinoid synthase gene is produced. The method can further comprise isolating the Cannabinoid synthase produced by the cell. In another aspect, the invention is directed to a Cannabinoid synthase gene produced by the method.

In yet another aspect, the invention is directed to a method of detecting a Cannabinoid in a sample comprising detecting the nucleic acid sequences described herein in the sample, wherein if the nucleic acid is detected, then a Cannabinoid is detected in the sample. The invention also encompasses a method of detecting Cannabis in a sample comprising detecting the polypeptides provided herein, wherein if the polypeptide is detected, then a Cannabinoid is detected in the sample.

In still other aspects, the invention is directed to a method of detecting one or more cannabinoid genes in a Cannabis plant. The method comprises contacting all or a portion of a genomic sequence of the Cannabis plant with one or more primers that are complementary to SEQ ID NO: 3, SEQ ID NO: 5, SEQ ID NO: 7, SEQ ID NO: 9 or a combination thereof, thereby producing a reaction mixture. The reaction mixture is maintained under conditions in which one or more sequences in the genomic sequence of the Cannabis plant that are complementary to one or more of the primers hybridize to the one or more primers. The one or more sequences that hybridize to the one or more primers are amplified, thereby producing one or more amplicons; and all or a portion of the sequence of the one or more amplicons is determined, thereby detecting one or more cannabinoid genes in the Cannabis plant. The method can further comprise quantifying the one or more Cannbinoid genes; measuring the Cannabinoid messenger ribonucleic acid (mRNA) of the plant, detecting whether fungal nucleic acid, bacterial nucleic acid, or a combination thereof is present in the plant; quantifying the fungal nucleic acid, bacterial nucleic acid, or a combination thereof if fungal nucleic acid, bacterial nucleic acid, or a combination thereof is present; and/or comparing the quantified fungal nucleic acid, bacterial nucleic acid, or a combination thereof to the quantified cannabinoid nucleic acid.

### BRIEF DESCRIPTION OF THE DRAWINGS

FIG. 1 shows the preliminary 2× assembly of 750 bp 454 GS FLX+ reads in the THC synthase gene.

FIGS. 2A-2B show a hairpin sequence (SEQ ID NO: 11) of a putative miniature P element inverted repeat family (mPIF) transposon sequence 5′ to the gene in the Sativa assembly.

FIG. 3 shows the target site for PIF insertion (Zhange et al., PNAS, 98(22):12572-12577 (2001) and the cannabis sativa gene for tetrahydrocannabinolic acid synthase (SEQ ID NO: 4).

FIG. 4 shows a Multiple Sequence Alignment and amino acid confirmation of MGC-s3 or LA_Contig#34396 vs PK contig #PK_{—}23203.1 (LA_contig34396_ORF_THCAS_like_{—}3 (SEQ ID NO: 6); PK23203.1_THCAS_like_{—}3 (SEQ ID NO: 16); CD_contig27237_ORF_THCAS_like_{—}3 (SEQ ID NO: 17); THC-Synthase_translation (SE QID NO: 18); Consensus (SEQ ID NO: 19)).

FIGS. 5A-5C shows a Multiple Sequence Alignment and conservation charts of peptide sequences from LAC, CD, PK and Mexican or “CSA” sequences. One can see divergent 5′ and 3′ ends with internal I→T changes from LAC & PK to CD&CSA at position 287 (FIG. 5C). Several internal amino acid changes can be seen with Sativa to Indica alignments in FIG. 5B. LAC & PK are Indica dominant and CD&CSA are Sativa dominant.

(FIG. 5A: LA_contig20041_ORF_THCAS_llike_{—}1 (SEQ ID NO: 20); PK20093.1_THCAS_like_{—}1 (SEQ ID NO: 21); THC_Synthase_translation (SEQ ID NO: 22); Consensus (SEQ ID NO: 23))

(FIG. 5B: LA_contig32071_ORF_THCAS_like_{—}2 (SEQ ID NO: 24); CD_contig32295_ORF_THCAS_like_{—}2 (SEQ ID NO: 25); PK09375.1_THCAS_like_{—}2 (SEQ ID NO: 26); THC_Synthase_translation (SEQ ID NO: 22); Consensus (SEQ ID NO: 27))

(FIG. 5C: LA_contig20817_ORF_THCAS_like_{—}4 (SEQ ID NO: 28); PK11708.1_THCAS_like_{—}4; THC_synthase-translation (SE QID NO: 22); Consensus (SEQ ID NO: 30))

FIG. 5D shows a Nucleic Acid multiple sequence alignments and conservation charts of many of the other THC-Like sequences in the LA confidential assembly with homology to THCA synthase, Purple Kush “PK” and Chemdawg “CD” closest contigs.

(THC Synthase (SEQ ID NO: 31); LA_contig-60432 (SEQ ID NO: 32): LA_contig_{—}20041 (SEQ ID NO: 33); LA_contig_{—}23755 (SEQ ID NO: 34); CBD_Synthase (SEQ ID NO: 35); LA_contig_{—}27956 (SEQ ID NO: 36); LA_contig_{—}46083 (SEQ ID NO: 37); LA_contig_{—}24266 (SEQ ID NO: 38); LA_contig_{—}86540 (SEQ ID NO: 39); LA_contig_{—}66523 (SEQ ID NO: 40); CD_contig_{—}27237_rev (SEQ ID NO: 41); PK_RNA_{—}23203.1 (SEQ ID NO: 42); LA_contig_{—}54324 (SEQ ID NO: 43); LA_contig_{—}163104 (SEQ ID NO: 44); Consensus (SEQ ID NO: 45))

FIG. 6A-6D show the nucleotide sequences of contig #20041 (SEQ ID NO: 3), contig #34396 (SEQ ID NO: 5), contig #32071 (SEQ ID NO: 7) and contig #20817 (SEQ ID NO: 9).

FIG. 7A-7D show the amino acid sequences of contig #20041 (SEQ ID NO: 4), contig #34396 (SEQ ID NO: 6), contig #32071 (SEQ ID NO: 8) and contig #20817 (SEQ ID NO: 10).

### DETAILED DESCRIPTION OF THE INVENTION

In recent years the pharmacology related to medicinal cannabis use has been transformed with the discovery of the human endocannabinoid pathways and the endogenous human neurotransmitter Anandamide (Devane et al. 1992, Science, 258(5090):1946-1949; Fride and Mechoulam 1993, Eur J Pharmacol, 231(2):401-409). Two human G-Protein coupled receptors (GPCRs) known as CB1 and CB2 have been extensively characterized and are encoded by CNR1 and CNR2 genes on chromosome 6 and 1 respectively. Three other GPCRs (GPR55, GPR18 and GPR119) are showing evidence as other potential endocannabinoid receptors (Begg et al. 2005, Pharmacol Ther, 106(2):133-145; Brown 2007, Br J Pharac, 152(2):567-575). Eighty-five phyto-cannabinoids have been discovered in the Cannabis plant (El-Alfy et al., Pharmacol Biochem Behav 95(4):434-442). Only one is known to be independently psychoactive (tertrahydrocannabinol or THC). Non-psychoactive cannabinoids like cannabidiol (CBD) and cannabidiolic acid (CBDA) have shown impressive medical benefits as it pertains to tumor specific apoptosis in 9 different cancer types (Guzman 2003, Nat Rev Ca, 3(10):745-755), pain management via cox-2 inhibition (Takeda et al. 2008, Drug Meatb Dispos 36(9):1917-1921), effectiveness with antiemesis in HIV or chemotherapy related nausea and improved muscle spasm control in patients with MS (Sarfaraz et al. 2008, Ca Res 68(2):339-342; Lakhan and Rowland 2009, BMC Neurol, 9:59). In addition the FDA has approved the use of Dronabinol and Nabilone for glaucoma. Combined with an extremely low therapeutic index, these reported medical benefits have resulted in a “compassionate use exemption” with 16 states and the District of Columbia decriminalizing medical use of cannabis in the United States and pharmaceutical companies actively investing in cannabinoid research. This has resulted in approved cannabinoid therapeutics such as Marinol™ and Sativex™.

Due in part to recreational demand, the cannabis plant has been selectively bred in the last 30 years to express very high THC levels (above 20% in the flower weight) (Miller Coyle et al. 2003, Croat Med J, 44(3):315-321). This has come at the cost of most plants available today having very low CBD content (below 1% flower weight) and considerable interest in the genetics controlling chemotype (Kojoma et al. 2006). To this end, De Meijer et al have demonstrated that the cannabinoid contents are under strict genetic control and can be predicted from DNA sequence information before the plant has expressed active compounds (de Meijer et al. 2003, Genetics, 163(1):335-346). The De Meijer study utilized PCR and Sanger sequencing to genotype CBD synthase and THC synthase in many drug and fiber strains but has stimulated many questions in regards to the genetics controlling the other 83 cannabinoids.

In addition to cannabinoids, the plant is reported to have up to 140 terpenes (Ross and ElSohly 1996, J Natl Prod, 59(1):49-51) (ElSohly 2007, Marijuana abd the cannabinoids.Human Press, Totowa, N.J.) at least one of which (Beta-caryophyllene) is reported to be a volatile CB2 receptor agonist (Gertsch et al. 2008, Proc Natl Acad Sci, USA, 105(26):9099-9104) with anti-inflammatory effects.

As described herein, using the efficiency of next generation sequencing, a draft de novo reference sequence for the Cannabis (C.) Sativa and C. Indica genomes has been generated. This provides for the sequencing and resequencing of many more cannabis cultivars to better understand the diversity of the genes encoding the cannabinoid and terpene synthesis or the “cannabinome”. In addition, as shown herein, the LAC Indica assembly herein had four full length contiguous sequences, referred to herein as “contigs” (Contigs #20041 (SEQ ID NOS: 3 and 4), #32071 (SEQ ID NOS: 7 and 8), #34396 (SEQ ID NOS: 5 and 6), #20817 (SEQ ID NOS: 9 and 10) with homology to THCA and CBDA synthases and 10 partially homologous contigs with truncated ORFs. The full length contig, in particular, #34396, 81% sequence similarity to both, was highly expressed in the PK Indica RNA-Seq data but was absent from the PK Indica Cansat3 genomic assembly.

Accordingly, in one aspect the invention is directed to an (one or more) isolated sequence (e.g., nucleic acid sequence, DNA, RNA, genomic sequence, polypeptide, protein) of a Cannabis genome.

In a particular aspect, the invention is directed to an isolated nucleic acid comprising SEQ ID NO: 1 (Cannabis sativa genome). In another particular aspect, the invention is directed to an isolated nucleic acid comprising SEQ ID NO: 2 (Cannabis indica genome). In other aspects, the invention is directed to an isolated sequence that has about (at least about, at least) 80%, 81%, 82%, 83%, 84%, 85%, 86%, 97%, 88%, 89%, 90%, 91%, 92%, 93%, 94%, 95%, 96%, 97%, 98%, of 99% to SEQ ID NO:1 and SEQ ID NO: 2.

In another aspect, the invention is directed to a nucleic acid comprising a nucleotide sequence that has about 82% to SEQ ID NO: 3, SEQ ID NO: 5, SEQ ID NO: 7 or SEQ ID NO: 9 or a portion thereof that encodes a biologically active cannabinoid synthase, or a complement thereof. In a particular aspect, the invention is directed to nucleic acid comprising SEQ ID NO: 3, SEQ ID NO: 5, SEQ ID NO: 7 or SEQ ID NO: 9 or a portion thereof that encodes a biologically active cannabinoid synthase, or a complement thereof. In other aspects, the invention is directed to an isolated sequence that has about (at least about; at least) 82%, 83%, 84%, 85%, 86%, 97%, 88%, 89%, 90%, 91%, 92%, 93%, 94%, 95%, 96%, 97%, 98%, of 99% to SEQ ID NOS: 3, 5, 7 or 9.

In another aspect, the invention is directed to a polypeptide comprising an amino acid sequence that has about 67% identity to SEQ ID NO: 4, SEQ ID NO: 6, SEQ ID NO: 8 or SEQ ID NO: 10 or a biologically active portion thereof, such as a biologically active portion that functions as a cannabinoid synthase. In a particular aspect, the invention is directed to a polypeptide comprising SEQ ID NO: 4, SEQ ID NO: 6, SEQ ID NO: 8 or SEQ ID NO: 10 or a biologically active portion thereof, such as a biologically active portion that functions as a cannabinoid synthase. In other aspects, the invention is directed to an isolated sequence that has about (at least about; at least) 67%, 68%, 69%, 70%, 71%, 72%, 73%, 74%, 75%, 76%, 77%, 78%, 79%, 80%, 81%, 82%, 83%, 84%, 85%, 86%, 97%, 88%, 89%, 90%, 91%, 92%, 93%, 94%, 95%, 96%, 97%, 98%, of 99% to SEQ ID NOS: 3, 5, 7 or 9.

As will be apparent to those of sill in the art, all or a portion of a biologically active cannabinoid synthase is a full length or portion of a full length cannabinoid synthase that has one or more activities of a cannabinoid synthase (e.g., atalyses the oxidocyclization of cannabigerolic acid to cannabidiolic acid).

Other aspects of the invention include an antibody that specifically binds one or more polypeptides described herein. antibody or antigen binding fragment thereof that specifically binds to all or a portion of polypeptides having the amino acid sequence of SEQ ID NOs: 4, 6, 8, and/or 10. That is, the antibody can bind to all of the polypeptide of from about 8 amino acids to about 450 amino acids of the polypeptide. In particular embodiments, the antibody can bind to about 10, 25, 50, 75, 100, 125, 150, 175, 200, 225, 250, 275, 300, 325, 350, 375, 400, or 425 amino acids of the polypeptide.

As used herein, the term “specific” when referring to an antibody-antigen interaction, is used to indicate that the antibody can selectively bind to the polypeptide. In one embodiment, the antibody inhibits the activity of the polypeptide. An antibody that is specific for polypeptides described herein is a molecule that selectively binds to the polypeptide but does not substantially bind to other molecules in a sample, e.g., in a biological sample a Cannabis plant. The term “antibody,” as used herein, refers to an immunoglobulin or a part thereof, and encompasses any polypeptide comprising an antigen-binding site regardless of the source, method of production, and other characteristics. The term includes but is not limited to polyclonal, monoclonal, monospecific, polyspecific, humanized, human, single-chain, chimeric, synthetic, recombinant, hybrid, mutated, conjugated and CDR-grafted antibodies. The term “antigen-binding site” refers to the part of an antibody molecule that comprises the area specifically binding to or complementary to, a part or all of an antigen. An antigen-binding site may comprise an antibody light chain variable region (VL) and an antibody heavy chain variable region (VH). An antigen-binding site may be provided by one or more antibody variable domains (e.g., an Fd antibody fragment consisting of a VH domain, an Fv antibody fragment consisting of a VH domain and a VL domain, or an scFv antibody fragment consisting of a VH domain and a VL domain joined by a linker).

The various antibodies and portions thereof can be produced using known techniques (Kohler and Milstein, Nature 256:495-497 (1975); Current Protocols in Immunology, Coligan et al., (eds.) John Wiley & Sons, Inc., New York, NY (1994); Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0 451 216 B1; and Padlan, E. A. et al., EP 0 519 596 A1; Newman, R. et al., BioTechnology, 10: 1455-1460 (1992); Ladner et al., U.S. Pat. No. 4,946,778; Bird, R. E. et al., Science, 242: 423-426 (1988)).

Also encompasses by the inventions are vectors comprising the nucleic acid sequences provided herein and cells comprising the vectors. As will be apparent to those of skill in the art a number of cells and/or vectors can be used in conjunction with the nucleic acid sequences provided herein. For example, a suitable plant cell includes a Cannabis plant cell and a suitable vector includes an agrobacterium vector.

In another aspect, the invention is directed to a method of producing a Cannabinoid synthase comprising maintaining a cell comprising a vector comprising the nucleic acid sequences provided herein under conditions in which the Cannabinoid synthase gene is produced. The method can further comprise isolating the Cannabinoid synthase produced by the cell. In another aspect, the invention is directed to a Cannabinoid synthase gene produced by the method.

In yet another aspect, the invention is directed to a method of detecting a Cannabinoid in a sample comprising detecting the nucleic acid sequences described herein in the sample, wherein if the nucleic acid is detected, then a Cannabinoid is detected in the sample. The invention also encompasses a method of detecting Cannabis in a sample comprising detecting the polypeptides provided herein, wherein if the polypeptide is detected, then a Cannabinoid is detected in the sample. The sample can be a plant sample (e.g., root tissue, leaf tissue) and/or a mammalian sample such as tissue (e.g. skin, hair), or fluid (e.g., urine, blood).

In still other aspects, the invention is directed to a method of detecting one or more cannabinoid genes in a Cannabis plant. The method comprises contacting all or a portion of a genomic sequence of the Cannabis plant with one or more primers that are complementary to SEQ ID NO: 3, SEQ ID NO: 5, SEQ ID NO: 7, SEQ ID NO: 9 or a combination thereof, thereby producing a reaction mixture. The reaction mixture is maintained under conditions in which one or more sequences in the genomic sequence of the Cannabis plant that are complementary to one or more of the primers hybridize to the one or more primers. The one or more sequences that hybridize to the one or more primers are amplified, thereby producing one or more amplicons; and all or a portion of the sequence of the one or more amplicons is determined, thereby detecting one or more cannabinoid genes in the Cannabis plant.

The method can further comprise quantifying the one or more Cannbinoid genes. In addition, the method can further comprise measuring the Cannabinoid messenger ribonucleic acid (mRNA) of the plant.

In a particular aspect, the method can further comprise detecting whether fungal nucleic acid, bacterial nucleic acid, or a combination thereof is present in the plant. As will be appreciated by those of skill in the art, if fungal nucleic acid, bacterial nucleic acid, or a combination thereof is present, then the fungal nucleic acid, bacterial nucleic acid or the combination thereof can also be quantified. The method can further comprise comparing the quantified fungal nucleic acid, bacterial nucleic acid, or a combination thereof to the quantified cannabinoid nucleic acid.

As will be apparent to those of skill in the art a number of methods can be used to detect and/or quantify one or more cannabinoid genes in a Cannabis plant such as polymerase chain reaction (PCR; quantitative PCR), real time PCR (rtPCR), and/or reverse transcription PCR. In addition a variety of methods can be used to detect and/or quantify bacterial and/or fungal nucleic acid in a Cannabis plant (e.g., SEQ™ Bacterial and Fungal Detection System, Life Technologies).

As will also be appreciated by those of skill in the art, the Cannabionoid, fungal and/or bacterial content can be compared to a control. Any suitable control can be used. For example, a suitable control can be established by assaying one or more (e.g., a large sample of) plants which do and/or do not have a Cannabinoid gene and using a statistical model to obtain a control value (standard value; known standard). See, for example, models described in Knapp, R. G. and Miller M. C. (1992) Clinical Epidemiology and Biostatistics, William and Wilkins, Harual Publishing Co. Malvern, Pa., which is incorporated herein by reference. Thus, as used herein, a “control” or “known standard” can to an amount and/or distribution characteristic of an plant that does or does not not have a cannbinoid gene.

As shown herein, sequencing of the Cannabis sativa genome revealed that the THC synthase gene has replicated itself throughout the genome via a mobile genetic element also referred to herein as a transposable element. As used herein, mobile genetic element or transposable element are elements or regions in a sequence that allow replication and insertion of a sequence into one or more additional places in a sequence such as a genomic sequence (see Jiang, N., et al., Nature, 42:163-167 *2003); Zhang, X., et al., PNAS, 98(22):12572-12577 (2001); Wessler, S., Miniature Inverted-repeat Transposable Elements (MITEs) and their Relationship with Established DNA Transposons, University of Georgia, Dept. Botany and Genetics, Athens, Ga., all of which are incorporated herein by reference).

Knowing this genome is tolerant of the copia and miniature inverted-repeat transposable elements (MITE) replication machinery enables the use of these sequences to replicate other desired synthase genes throughout the plant. Of particular interest is the CBD synthase gene that produces the anti-cancer compound cannabidiol.

Knowledge of the transposon systems which are tolerated by this species opens up avenues for improving the production of other cannabinoids. Specifically, the use of these transposons to increase the % CBD (cannanbidiol) expressed would aid in, for example, fighting cancer. More specifically, synthesizing a DNA fragment which has the leader sequence identical to the THC synthase gene and its transposon signal where the THC synthase gene is replaced with CBD synthase one could then use Agrobacteria or other pant transfection tools such as Gene Gun to introduce many more CBD synthase genes into the plant. This would result in a plant that expresses increased levels of CBD.

Accordingly, in another aspect, the invention is directed to a method of increasing the copy number of one or more sequences in a Cannabis genome comprising operably linking the one or more sequences to one or more mobile genetic elements, thereby increasing the copy number of one or more sequences in a Cannabis. In yet another aspect, the invention provides methods of introducing such sequences operably linked to one or more mobile genetic elements into a plant (e.g., a Cannabis plant) using, for example, a plant transfection tool, e.g., Agrobacteria, and maintaining the plant under conditions in which the copy number of the one or more sequences is increased in the plant (under conditions in which the expression of polypeptide encoded by the sequence is increased in the plant, for example, as compared to a plant which does not comprise the sequence operably linked to the mobile genetic element). The invention is also directed to plants produced by the methods.

Thus, examples of sequences whose copy number could be increased include sequences that encode one or more polypeptides involved in the biosynthesis of one or more cannabinoids, and/or one or more terpenes. Specific examples include sequences that encode a Cannabidiol (CBD) synthase, a Cannabichromene (CBC) synthase or other Cannabinoids in place of THC synthase, olivetol acid synthase, divarinic acid synthase limonene synthase, and alpha pinene synthase. Specific examples of other such sequences include the following:

example of a sequence that encodes an Olivetol Synthase >gi|171363646|dbj|AB164375.1| Cannabis sativa OLS mRNA for olivetol synthase, complete cds

(SEQ ID NO: 13)
ATGAATCATCTTCGTGCTGAGGGTCCGGCCTCCGTTCTCGCCATTGGCAC
CGCCAATCCGGAGAACATTT
TATTACAAGATGAGTTTCCTGACTACTATTTTCGCGTCACCAAAAGTGAA
CACATGACTCAACTCAAAGA
AAAGTTTCGAAAAATATGTGACAAAAGTATGATAAGGAAACGTAACTGTT
TCTTAAATGAAGAACACCTA
AAGCAAAACCCAAGATTGGTGGAGCACGAGATGCAAACTCTGGATGCACG
TCAAGACATGTTGGTAGTTG
AGGTTCCAAAACTTGGGAAGGATGCTTGTGCAAAGGCCATCAAAGAATGG
GGTCAACCCAAGTCTAAAAT
CACTCATTTAATCTTCACTAGCGCATCAACCACTGACATGCCCGGTGCAG
ACTACCATTGCGCTAAGCTT
CTCGGACTGAGTCCCTCAGTGAAGCGTGTGATGATGTATCAACTAGGCTG
TTATGGTGGTGGAACCGTTC
TACGCATTGCCAAGGACATAGCAGAGAATAACAAAGGCGCACGAGTTCTC
GCCGTGTGTTGTGACATAAT
GGCTTGCTTGTTTCGTGGGCCTTCAGAGTCTGACCTCGAATTACTAGTGG
GACAAGCTATCTTTGGTGAT
GGGGCTGCTGCGGTGATTGTTGGAGCTGAACCCGATGAGTCAGTTGGGGA
AAGGCCGATATTTGAGTTGG
TGTCAACTGGGCAAACAATCTTACCAAACTCGGAAGGAACTATTGGGGGA
CATATAAGGGAAGCAGGACT
GATATTTGATTTACATAAGGATGTGCCTATGTTGATCTCTAATAATATTG
AGAAATGTTTGATTGAGGCA
TTTACTCCTATTGGGATTAGTGATTGGAACTCCATATTTTGGATTACACA
CCCAGGTGGGAAAGCTATTT
TGGACAAAGTGGAGGAGAAGTTGCATCTAAAGAGTGATAAGTTTGTGGAT
TCACGTCATGTGCTGAGTGA
GCATGGGAATATGTCTAGCTCAACTGTCTTGTTTGTTATGGATGAGTTGA
GGAAGAGGTCGTTGGAGGAA
GGGAAGTCTACCACTGGAGATGGATTTGAGTGGGGTGTTCTTTTTGGGTT
TGGACCAGGTTTGACTGTCG
AAAGAGTGGTCGTGCGTAGTGTTCCCATCAAATATTAA

example of a sequence that encodes a Limonene synthase >gi|112790154|gb|DQ839404.1| Cannabis sativa (−)-limonene synthase mRNA, complete cds

(SEQ ID NO: 14)
ATGCAGTGCATAGCTTTTCACCAATTTGCTTCATCATCATCCCTCCCTAT
TTGGAGTAGTATTGATAATC
GTTTTACACCAAAAACTTCTATTACTTCTATTTCAAAACCAAAACCAAAA
CTAAAATCAAAATCAAACTT
GAAATCGAGATCGAGATCAAGTACTTGCTACTCCATACAATGTACTGTGG
TCGATAACCCTAGTTCTACG
ATTACTAATAATAGTGATCGAAGATCAGCCAACTATGGACCTCCCATTTG
GTCTTTTGATTTTGTTCAAT
CTCTTCCAATCCAATATAAGGGTGAATCTTATACAAGTCGATTAAATAAG
TTGGAGAAAGATGTGAAAAG
GATGCTAATTGGAGTGGAAAACTCTTTAGCCCAACTTGAACTAATTGATA
CAATACAAAGACTTGGAATA
TCTTATCGTTTTGAAAATGAAATCATTTCTATTTTGAAAGAAAAATTCAC
CAATAATAATGACAACCCTA
ATCCTAATTATGATTTATATGCTACTGCTCTCCAATTTAGGCTTCTACGC
CAATATGGATTTGAAGTACC
TCAAGAAATTTTCAATAATTTTAAAAATCACAAGACAGGAGAGTTCAAGG
CAAATATAAGTAATGATATT
ATGGGAGCATTGGGCTTATATGAAGCTTCATTCCATGGGAAAAAGGGTGA
AAGTATTTTGGAAGAAGCAA
GAATTTTCACAACAAAATGTCTCAAAAAATACAAATTAATGTCAAGTAGT
AATAATAATAATATGACATT
AATATCATTATTAGTGAATCATGCTTTGGAGATGCCACTTCAATGGAGAA
TCACAAGATCAGAAGCTAAA
TGGTTTATTGAAGAAATATATGAAAGAAAACAAGACATGAATCCAACTTT
ACTTGAGTTTGCCAAATTGG
ATTTCAATATGCTGCAATCAACATATCAAGAGGAGCTCAAAGTACTCTCT
AGGTGGTGGAAGGATTCTAA
ACTTGGAGAGAAATTGCCTTTCGTTAGAGATAGATTGGTGGAGTGTTTCT
TATGGCAAGTTGGAGTAAGA
TTTGAGCCACAATTCAGTTACTTTAGAATAATGGATACAAAACTCTATGT
TCTATTAACAATAATTGATG
ATATGCATGACATTTATGGAACATTGGAGGAACTACAACTTTTCACTAAT
GCTCTTCAAAGATGGGATTT
GAAAGAATTAGATAAATTACCAGATTATATGAAGACAGCTTTCTACTTTA
CATACAATTTCACAAATGAA
TTGGCATTTGATGTATTACAAGAACATGGTTTTGTTCACATTGAATACTT
CAAGAAACTGATGGTAGAGT
TGTGTAAACATCATTTGCAAGAGGCAAAATGGTTTTATAGTGGATACAAA
CCAACATTGCAAGAATATGT
TGAGAATGGATGGTTGTCTGTGGGAGGACAAGTTATTCTTATGCATGCAT
ATTTCGCTTTTACAAATCCT
GTTACCAAAGAGGCATTGGAATGTCTAAAAGACGGTCATCCTAACATAGT
TCGCCATGCATCGATAATAT
TACGACTTGCAGATGATCTAGGAACATTGTCGGATGAACTGAAAAGAGGC
GATGTTCCTAAATCAATTCA
ATGTTATATGCACGATACTGGTGCTTCTGAAGATGAAGCTCGTGAGCACA
TAAAATATTTAATAAGTGAA
TCATGGAAGGAGATGAATAATGAAGATGGAAATATTAACTCTTTTTTCTC
AAATGAATTTGTTCAAGTTT
GCCAAAATCTTGGTAGAGCGTCACAATTCATATACCAGTATGGCGATGGA
CATGCTTCTCAGAATAATCT
ATCGAAAGAGCGCGTTTTAGGGTTGATTATTACTCCTATCCCCATGTAA

example of a sequence that encodes an Alpha Pinene synthase >gi|112790156|gb|DQ839405.1| Cannabis sativa (+)-alpha-pinene synthase mRNA, complete cds

(SEQ ID NO: 15)
ATGCATTGCATGGCTGTTCGCCATTTCGCTCCATCGTCATCGCTCTCCAT
ATTTTCGAGTACTAATATTA
ATAATCATTTTTTTGGTAGAGAAATTTTTACACCAAAAACATCTAATATT
ACAACAAAAAAATCAAGATC
AAGACCTAATTGCAATCCAATCCAATGTAGTTTGGCCAAAAGCCCTAGTA
GTGATACTAGTACAATTGTT
AGAAGATCAGCCAACTATGATCCTCCCATTTGGTCTTTTGATTTCATTCA
GTCTCTTCCATGCAAATATA
AGGGAGAACCCTATACAAGTCGATCGAATAAGCTAAAAGAAGAAGTGAAA
AAGATGTTAGTTGGAATGGA
AAACTCTTTAGTCCAACTTGAGTTGATTGATACATTACAAAGACTTGGAA
TATCTTATCATTTTGAGAAT
GAAATCATTTCTATTTTGAAAGAATATTTCACTAATATTAGTACTAATAA
AAACCCTAAATATGATTTAT
ATGCCACTGCTCTCGAATTTAGGCTTTTACGCGAATATGGATATGCAATA
CCTCAAGAAATATTTAATGA
TTTTAAGGACGAGACGGGAAAGTTCAAAGCGAGTATTAAAAATGATGATA
TTAAGGGAGTATTGGCTTTA
TATGAAGCTTCATTCTATGTGAAAAATGGTGAAAATATTTTGGAGGAAGC
TAGGGTTTTCACAACAGAAT
ATCTCAAAAGATATGTAATGATGATTGATCAAAACATAATATTAAATGAT
AATATGGCAATATTAGTGAG
ACATGCCTTGGAGATGCCACTTCATTGGAGGACTATAAGAGCAGAAGCTA
AGTGGTTCATTGAAGAATAT
GAGAAGACACAAGACAAGAATGGCACTTTGCTTGAATTTGCGAAATTGGA
TTTCAACATGCTTCAATCAA
TATTTCAAGAAGATCTAAAACATGTCTCGAGGTGGTGGGAACATTCTGAG
CTTGGAAAGAATAAAATGGT
TTATGCTAGAGATAGATTGGTAGAGGCTTTTCTATGGCAGGTTGGAGTAA
GATTTGAGCCACAATTCAGC
CACTTTAGGAGAATATCTGCAAGAATATATGCTCTAATTACAATCATAGA
TGACATATATGATGTGTATG
GAACATTGGAAGAGTTAGAGCTTTTCACCAAGGCTGTTGAGAGATGGGAT
GCGAAGACCATACACGAGTT
ACCAGATTATATGAAGTTGCCTTTCTTTACTTTATTTAACACCGTAAATG
AAATGGCGTATGATGTATTA
GAAGAGCATAATTTTGTCACCGTTGAATACCTCAAGAACTCGTGGGCAGA
GTTATGTAGGTGCTATTTGG
AAGAGGCAAAATGGTTCTATAGCGGATACAAACCAACCTTGAAAAAATAT
ATTGAGAACGCCTCGCTTTC
AATAGGAGGACAAATTATTTTTGTATATGCTTTTTTCTCTCTTACAAAGT
CCATAACAAACGAGGCCTTA
GAGTCCTTGCAAGAGGGTCATCACGCTGCATGTCGCCAAGGATCCTTAAT
GTTACGACTTGCAGATGATC
TAGGAACATTGTCGGATGAAATGAAAAGAGGCGATGTTCCTAAATCAATT
CAATGTTATATGCACGATAC
TGGTGCTTCTGAAGATGAAGCTCGTGAGCACATCAAATTTTTGATAAGTG
AAATATGGAAGGAGATGAAT
GATGAAGATGAATATAACTCTATTTTCTCTAAAGAGTTTGTTCAAGCTTG
CAAAAATCTTGGTAGGATGT
CATTATTTATGTATCAACATGGAGATGGACATGCTTCTCAAGATAGCCAT
TCAAGGAAACGTATTTCAGA
TTTAATTATTAATCCTATTCCTTTATAA

In other aspects, the invention is directed to method of sequencing a genome of a target species within a genus, wherein the genome of the species within the genus vary by about 1 in about 100 bases. Next Generation sequencers drop the cost of sequencing genomes 100,000 fold by using one clever trick. They know what they looking for. The majority of these massively parallel short read (<400bp) sequencing systems are successful at sequencing humans because there is a reference genome to compare short reads to. Since the human genome is not very polymorphic only 1 in 1000 letters is different. This means that most reads from a Next Generation sequencer map to the genome perfectly and when there is a variant there is most likely only one in that 100bp read.

Each human genome sequenced on SOLiD or Illumina usually generates 4M SNPs and 400,000 deletion or insertion polymorphisms and 40,000 large copy number variations of structural variations larger than 1,000 bases. Since humans diverged so recently, we are mostly the same that makes resequencing the human genome a very easy analysis problem. One can load the 3 billion bases into RAM and scan every read across this index and find locations for where all the reads should be placed and regions where mutations occur with commodity hardware. This is described as an algorithmic problem that scales to N of the reads in the analysis. More reads=linearly more time but the reference genome is always hg19 (the human genome in genbank). This is all possible because the human genome project spent billions of dollar first making this reference with expensive tools that generate long reads.

This long read process is very different. When there is no reference genome to work with one must compare every read to all other reads so if you have 20 Million reads, the computation problem is now 20M reads×20M reads or 400 Trillion comparisons. This is called a N̂2 (N squared) problem as its not linear but multiplicative based on the read numbers. Some advancements in algorithms have made this an N log N problem by sorting reads and using small word sizes but this is still substantially more computationally intensive than resequencing and alignment to a reference. In other words this is computationally a much more difficult problem than matching reads to a 3Billion letter sequence. This is known as “de novo” sequencing as opposed to “resequencing” used for most humans today.

There are some examples of people using de novo assembly on humans despite its excessive costs as it is thought to be more thorough but this is still very bleeding edge in terms of its completeness next to re-alignment. Some have suggested to perform a hybrid approach to get the best of both methods.

With the costs of DNA sequencing plummeting the cost to perform the easier Re-alignment process is still at least half the cost a genomics experiment and de novo assembly is likely 90% of the cost of the sequencing project so efficient use of the computational architecture is now more important than cheaper sequencing methods.

Until now, cannabis has never had its entire genome sequenced. As shown herein, in sequencing Cannabis it was discovered that the polymorphism rate in the plant was 10× higher than in humans. This means the re-alignment problem needed to be re-invented to even work and enable a non de novo assembly approach. To this end, a method to generate not 1 reference sequence but 2 or more references was devised. PIn a particular aspect, 3 reference sequences, one for each of the known cultivars in the field are used. Cannabis has 3 known species; Sativa, Indica and Ruderalis. These 3 have been interbred and the strategy devised herein involved back crossing each of these strains to be pure species and then making a reference genome from each of them. By having 3 reference genomes the reads were aligned to all 3 references, variants were called on all 3 and a Venn Diagram of the variation within all there species were generated for novel strains being sequenced. This was computationally much cheaper than a full blown de novo assembly for each strain and provided important information, which a de novo assembly may miss as it leverages the information of what is already known about the plants and will be more tolerant to repeat structures.

In the method of sequencing a genome of a target species within a genus, wherein genomes of species within the genus vary by about 1 base in about 100 bases, the method comprises obtaining sequencing reads of the genome of the target species (e.g., using massively parallel sequencing), aligning the sequencing reads to at least two different reference sequences, wherein each reference sequence is a known sequence of a species within the genus; and obtaining a consensus of variation between the sequence of the target species and each reference sequence, thereby sequencing the genome of the target species. In a particular aspect, the sequencing reads are aligned to at least three reference sequences (e.g., Cannabis sativa, Cannabis indica, Cannabis ruderalis).

The genetics governing the synthesis of the 85 phyto-cannabinoids found in Cannabis Sativa L. are only known for the tetrahydrocannabinolic acid (THCA) and cannabidiolic acid (CBDA) synthase pathways. While, the Cannabis Sativa sequence of Purple Kush has recently been compared to hemp, less is known in regards to how each medicinal strain of cannabis may vary with respect to each other. To this end, presented herein is a de novo assembly of the medicinal plants Cannabis Sativa and Cannabis Indica. These diploid assemblies range in size from 300 Mb to 727 Mb, are 65% AT, and have mitochondrial genomes up to 415 Kb. Over 1.5 million SNVs for the Sativa genome, 925,602 SNVs for the Indica genome, and approximately 4M single nucleotide variants (SNVs) compared to the recently published Purple Kush, 30% of which are found in both our Sativa and Indica references, are detailed. These assemblies cover over 85% of the Cannabis RNA-seq sequence in genbank. Of particular interest is a copy number variation in the synthase genes responsible for cannabigerolic acid (CBGA) conversion to THCA. Also evident is flower to root differential expression of this expanded gene family and novel synthase homologs not found in the Purple Kush assembly. These data provide selective breeding strategies to alter medicinal expression.

Non-psychoactive cannabinoids like cannabidiol (CBD) and cannabidiolic acid (CBDA) exhibit evidence of tumor specific apoptosis in 9 different cancer cell types, pain management via cox-2 inhibition, effectiveness with antiemesis from chemotherapy, and enhanced muscle spasm control in patients with MS. Separately, the FDA has approved the use of cannabinoid drugs Dronabinol and Nabilone for chemotherapy related nausea and HIV related appetite stimulation. 84 other cannabinoids have been measured in Cannabis and their expression varies tremendously plant to plant. The pharmacology of cannabinoids has been transformed with the discovery of the human endocannabinoid pathways and the endogenous human neurotransmitters anandamide and 2-AG. Two human G-Protein coupled receptors (GPCRs) known as CB₁ and CB₂ have been extensively characterized and are encoded by CNR1 and CNR2 genes on chromosome 6 and 1, respectively. Mutations in these human receptor genes are associated with increased addiction and extreme body mass index. Three additional GPCRs (GPR55, GPR18 and GPR119) are showing evidence as potential endocannabinoid receptors. Combined with an extremely low therapeutic index, these reported medical benefits have resulted in a “compassionate use exemption” with 16 states and the District of Columbia decriminalizing medical use of cannabis in the United States for non-FDA approved “off label” indications. Despite the popular medicinal use, the genetics of the GPCR targets and genes governing the cannabinoid expression remain only partially characterized.

Due in part to prohibition, the cannabis plant has been selectively bred in the last 30 years to express very high tetrahydrocannabinol (THC) levels (above 20% in the flower weight). Due to THCA and CBDA synthase competition for their shared pathway precursor CBGA, this selective pressure has come at the cost of most strains available today containing very low cannabidiol (CBD) content (below 1% flower weight). This in turn has prompted considerable interest in the genetics controlling chemotype. To this end, others have demonstrated that the cannabinoid contents are under strict genetic control and can be predicted from DNA sequence information before the plant has expressed active compounds. This study has stimulated many questions in regards to the genetics controlling the other cannabinoids, as well as the 140 terpenes reportedly expressed in the plant. These terpenes also compete for an IPP cannabinoid precursor. At least one of these terpenes, (Beta-caryophyllene) is reported to be a volatile CB2 receptor agonist with anti-inflammatory effects.

Described herein is the generation of a draft de novo reference sequence for the C. Sativa and C. Indica genomes with a focus on resolving the high polymorphism rates in the synthase genes. This provides a view of drug type strain differences along with a complementary tool for many ongoing investigations in other cultivars.

### Exemplification

### EXAMPLE 1

### Methods

DNA was purified with Qiagen Mini and Maxi plant DNA purification Kits. Sativa cultivar “Chemdawg” and Indica cultivar “L.A. Confidential” were used as the first reference genomes (DNA Genetics). CBD and THC levels were measured with HPLC and GC analysis by Steep Hills Lab. Results were verified with Thin Layer Chromatography prior to sequencing (Montana Biotech). Sequencing of the Indica reference genome was accomplished with twelve 454 GS FLX+700bp runs delivering and an estimated 12× coverage. Genome sequencing and assembly was performed by the 454 Sequencing center in Branford CT with Newbler. The Sativa strain utilized a hybrid assembly approach with 100× of 2×100 ILMN HiSeq (651M reads, 131 Gb of PF filtered data) sequencing reads combined with an additional four 454 FLX 400 bp runs. These reads were assembled with CLCbio Genomics Workbench 4.7.1. High quality reads not mapping to the assembly were retained for separate de novo assembly.

To PCR or Sequence DNA from Cannabis, Plant DNA material was purified from the plant. 100-300 mg of dry plant material was first diced into fine plant fragments with a knife or razor. This material was then added to Qiagen Plant Lysis buffer or AP1 was added. 2× more lysis buffer than the manufacturer recommended was added as the plant flowers are very lipophilic. For each 1 g of plant material 10 ml of AP1 was added and heated to 65° C. for 10 minutes while inverting and vortexing for a minute every 3 minutes. Plant material was placed into an IKA turrax tissue homogenizer tube mixer prefilled with 5 ml of AP1 and vorterxed at top speed for 10 seconds and 2 minutes at 2000 rpm. Morter and Pescle homogenization with liquid nitrogen was used but yields can vary. With the exception of the 3× increased AP1, the rest of the protocol followed was according to Qiagens plant mini-prep volume suggestions (part number in 2011 is 69104) (increased everything 3× accordingly with the exception of the final elution step). Qiagen MaxiPrep columns can also be used to handle the increased 3× volume recommendation. Lower volumes showed lower yield as the plant oils seem to interfere with the prep but this was dependent on how dry the sample is. Fresh plant clippings used 2× volume recommendations and 1× delivered DNA. DNA purified with this method was predominantly more than 10,000 bases in length for 10 different cultivars according to E-Gel 1% gel analysis. Fragments could be larger due to the gels resolution.

After Qiagen isolation, DNA most likely didn't freeze do to glycols, terpenes and other pigments in the isolation. Use of Beckman Genomics Ampure was used to clean these samples up (formerly known as Agencourt Ampure). 100 ul of Ampure to 100 ul of sample instead of the Manufacturers instructions of 180 ul of Ampure to 100 ul of sample was used to save on reagents and keep the conditions within the volume of a 96 well plate and a 96 well magnet plate magnetic field.

Lower ratios of Ampure (50 ul to 100 ul) were tested and worked well. This lowered cost but quantitative yields across many cultivars may vary. This DNA was clean enough to freeze and used in most next generation sequencing library construction kits like the SPRIworks system from Beckman. Multiple different libraries can be made from fragment libraries to jumping libraries or even RNA libraries. Described below is the simplest library but those skilled in the art will know how to apply and RNA or DNA prep to a kit that converts this DNA or RNA to sequencable material. What is important is to be able to purify the DNA from a plant high in oil, cannabinoid and terpenes content to ensure it will be pure enough to be enzymatically active .

Fragment libraries are short (less than 1000 bases and usually less than 600 bp). To get DNA this small after isolation from a plant, a covaris or nebulization device from Life Technologies was used to shear the high molecular weight (HMW) DNA into smaller fragments that were amenable to the Next Generation Sequencers (Illumina, SOLiD, 454, Ion Torrent, Pacific Biosciences, Helicos and others).

Purified DNA was nebulized/sonicated/acoustic bombardment (Covaris Corp) or hydrodynamicaly sheared to break the DNA down to more managable pieces as large DNA acts like a viscous polymer which is difficult to manage and inefficient in ligation. Once HMW DNA was broken into smaller pieces, known sequences or “Primers” (also known as “Adaptors”) were added to both ends of the DNA fragment. These known sequence sites can be any sequence a person desires but are preferable sequences the popular DNA sequencing platforms utilize for sequencing. Once “Adapted” the distribution was measured with an Agilent Bioanalyzer or other gel eletrcophoresis device and decide if size selection is needed to narrow the library size distribution. The Agilent gel was size selected as its distribution was large but this is very dependent on the sequencing platform and strategy. The size range of DNA for sequencing was selected. It's preferable to have a very tight size distribution, e.g., much tighter than the initial HMW prep where fragments range from 50 bp to 1500 bp. A fraction of this material in the 300-400 bp range was collected and a Polymerase Chain Reaction performed to make many copies of the molecules in this size range. Once many copies were made they were put on a Next Generation Sequencer for Massively Parallel Sequencing. The fragment distribution for the sheared library DNA measured was obtained on an Agilent Bioanalyzer for the ChemDawg cultivar sequenced to over 350× coverage on the Illumina HiSeq 2000 platform by Beckman Genomics. The distribution after size selection and PCR was also obtained.

### Results

To address the polymorphism rate in the genome, a triple backcrossed pure Indica cultivar named LA Confidential (DNA Genetics, NL) was chosen to build a reference genome with over 12 million 454 GS FLX+ 750 bp reads (6.4 Gb). The genome was assembled with three different alignment stringencies on CLCbio workbench (0.8 or default, 0.9 and 0.95). N50 contigs of 1500-1600 bp and genome sizes ranging from 280 Mb to 303 Mb were obtained. An outbred Sativa cultivar known as “Chemdawg” was also sequenced with 131 Gb from Illumina's HiSeq platform with 2×100 reads from 250 bp inserts. 164M paired reads (single lane of 7) were assembled with the CLCbio workbench and resulted in N50s of 2.2 Kb and a genome size of 288 Mb.

To assess genome completeness, all Cannabis DNA sequence in Genbank were aligned to the Indica reference and significant blast hits for over 98.3% of the entries were found. Many of these entries were mRNA sequences and thus enriched for euchromatic sequence. To assess the heterochromatic coverage the number of reads (filtered of dots and polyclonals) not mapped in the varying assemblies was measured. These ranged from 9.8% of the reads at the default alignment stringency to 33% of the reads at the most stringent assembly conditions. To complement this all of the Sativa reads were mapped to the Indica references where non-unique sequence was left unmapped and only 22% of the reads were found to not map to the 0.95 stringent Indica reference. The Indica reads with the 0.9 mapping stringency were mapped back to the stringent Indica assemble and 14% of the reads were found to not map indicating a genome size of 346 Mb. Using the methods described by Xu et al (Xu et al. 2001, Natl Biotech, 29(8):73741) a 396 Mb genome size was estimated using the total kmer number/kmer volume of the Sativa assembly. This differs from prior published reports on the genome size (Sakamoto) of 1.4 pg per diploid genome but flow sorting technique can be very sensitive to GC content based on the stains used (Greilhuber 2005, Ann Bot, 95(1):91-98) and male plants are known to have larger genomes than female cannabis genome sequenced in this study. Reads that don't assemble have a GC content of Y % and consist of low complexity sequence.

To assess polymorphisms on a draft genome, reads to the consensus assemblies were remapped to look for single nucleotide polymorphisms (SNPs) and deletion/insertion polymorphisms (DIPs) (Indels). This produces heterozygous SNPs for self mappings but heterozygous and homozygous SNPs for cross cultivar mappings. As expected, the more outbred Sativa cultivar had more variation than the triple backcrossed Indica and both cultivars exhibited a high degree of polymorphism as compared to the variation content seen the human genome.

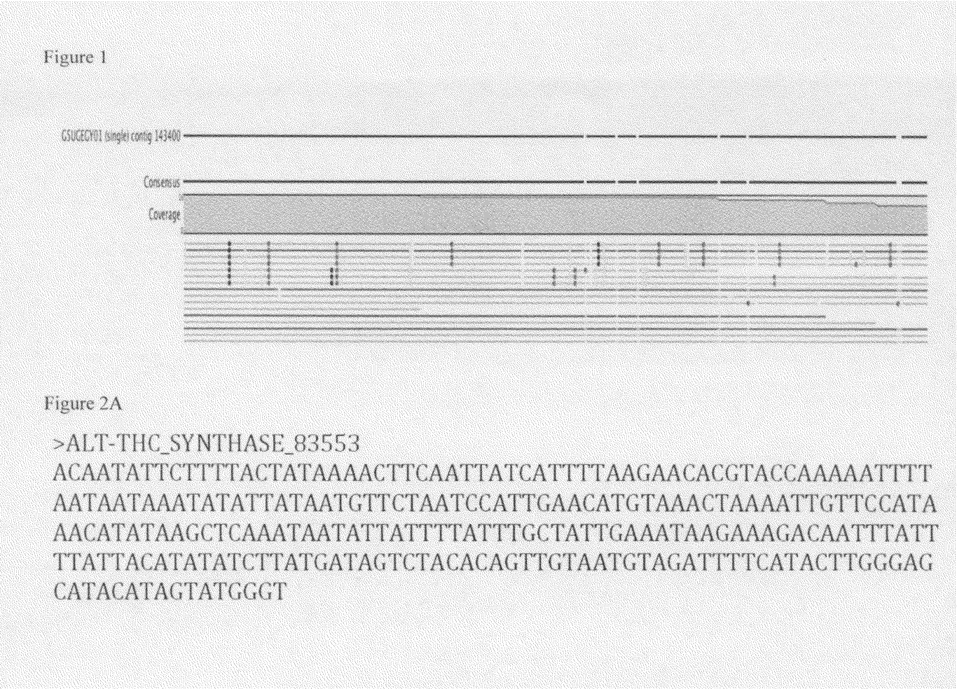

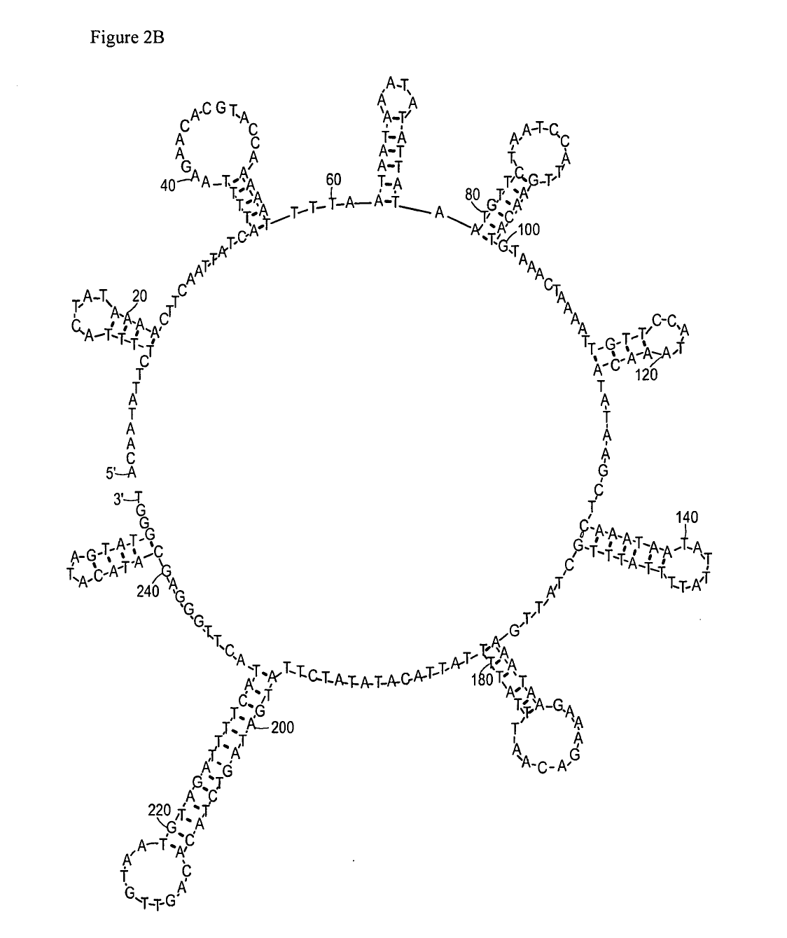

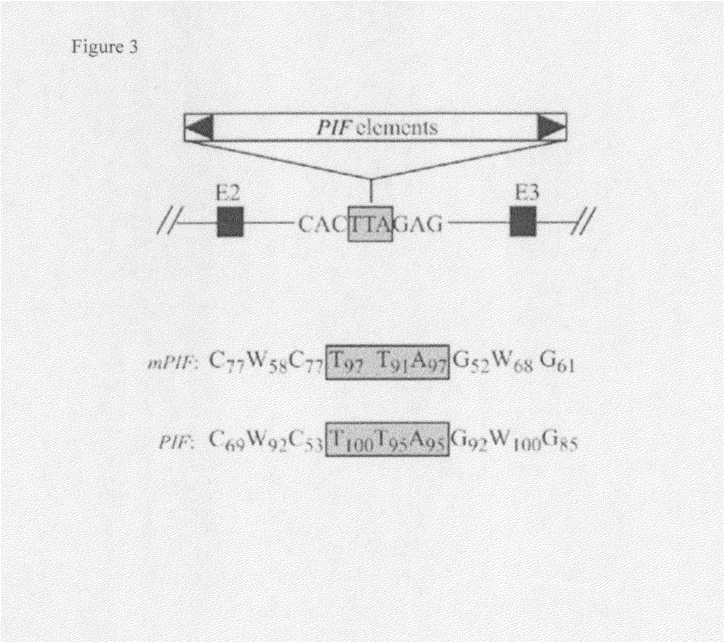

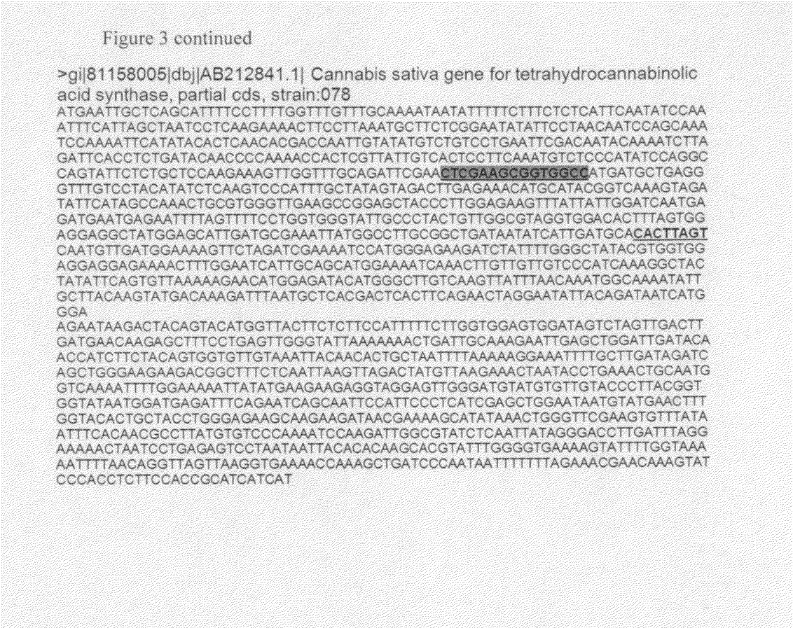

The THC synthase genes display a polymorphism rate closer to 5% perhaps explained by this being a gene governing the dominant phenotype monitored with selective breeding. With short reads alone, phasing the sequence to provide accurate amino acid prediction was challenging, however many SNPs in the THC synthase gene are nicely phased with the 750 bp 454 data. Evidence for a gene expansion can be seen in this data with the increased genome coverage in this location (FIG. 1). One can see more phased alleles than expected with a diploid plant. On the boundaries of this gene a sequence with homology to the mPIF transposon family (e value of 2e-6) was observed that likely explains the expansion. This region has coverage 100 fold higher than average and is likely an assembly knot but multiple 700 bp reads with THC synthase sequence read into the mPIF homologous sequence implying copies of THC synthase were in tight linkage with this putative transposable element. As with other mPIF transposons, a long inverted sequence is present 5′ to the THC synthase gene (FIG. 2B). The Hairpin seen using mFold in the putative mPIF transposon sequence 5′ to the gene in the Sativa Assembly. Also observed in the 454 sequence on reads which map to THC but have frayed high quality ends.

### >ALT-THC SYNTHASE 83553

(SEQ ID NO: 11)
ACAATATTCTTTTACTATAAAACTTCAATTATCATTTTAAGAACACGTAC
CAAAAATTTTAATAATAAATATATTATAATGTTCTAATCCATTGAACATG
TAAACTAAAATTGTTCCATAAACATATAAGCTCAAATAATATTATTTTAT
TTGCTATTGAAATAAGAAAGACAATTTATTTTATTACATATATCTTATGA
TAGTCTACACAGTTGTAATGTAGATTTTCATACTTGGGAGCATACATAGT
ATGGGT.

DNA sequence of the THCA synthase gene reported by Kojoma et al. Highlighted and underlined section, CTCGAAGCGGTGGCC, is the FAD binding domain. Highlighted region, CACTTAGT, is the mPIF signal described by Zhang et al. 2001 Proc Natl Acad Sci, USA 98(22):12572-12577 >gi|81158005|dbj|AB212841.1| Cannabis sativa gene for tetrahydrocannabinolic acid synthase, partial cds, strain:078

(SEQ ID NO: 12)
ATGAATTGCTCAGCATTTTCCTTTTGGTTTGTTTGCAAAATAATATTTTT
CTTTCTCTCATTCAATATCCAAATTTCATTAGCTAATCCTCAAGAAAACT
TCCTTAAATGCTTCTCGGAATATATTCCTAACAATCCAGCAAATCCAAAA
TTCATATACACTCAACACGACCAATTGTATATGTCTGTCCTGAATTCGAC
AATACAAAATCTTAGATTCACCTCTGATACAACCCCAAAACCACTCGTTA
TTGTCACTCCTTCAAATGTCTCCCATATCCAGGCCAGTATTCTCTGCTCC
AAGAAAGTTGGTTTGCAGATTCGAACTCGAAGCGGTGGCCATGATGCTGA
GGGTTTGTCCTACATATCTCAAGTCCCATTTGCTATAGTAGACTTGAGAA
ACATGCATACGGTCAAAGTAGATATTCATAGCCAAACTGCGTGGGTTGAA
GCCGGAGCTACCCTTGGAGAAGTTTATTATTGGATCAATGAGATGAATGA
GAATTTTAGTTTTCCTGGTGGGTATTGCCCTACTGTTGGCGTAGGTGGAC
ACTTTAGTGGAGGAGGCTATGGAGCATTGATGCGAAATTATGGCCTTGCG
GCTGATAATATCATTGATGCACACTTAGTCAATGTTGATGGAAAAGTTCT
AGATCGAAAATCCATGGGAGAAGATCTATTTTGGGCTATACGTGGTGGAG
GAGGAGAAAACTTTGGAATCATTGCAGCATGGAAAATCAAACTTGTTGTT
GTCCCATCAAAGGCTACTATATTCAGTGTTAAAAAGAACATGGAGATACA
TGGGCTTGTCAAGTTATTTAACAAATGGCAAAATATTGCTTACAAGTATG
ACAAAGATTTAATGCTCACGACTCACTTCAGAACTAGGAATATTACAGAT
AATCATGGGAAGAATAAGACTACAGTACATGGTTACTTCTCTTCCATTTT
TCTTGGTGGAGTGGATAGTCTAGTTGACTTGATGAACAAGAGCTTTCCTG
AGTTGGGTATTAAAAAAACTGATTGCAAAGAATTGAGCTGGATTGATACA
ACCATCTTCTACAGTGGTGTTGTAAATTACAACACTGCTAATTTTAAAAA
GGAAATTTTGCTTGATAGATCAGCTGGGAAGAAGACGGCTTTCTCAATTA
AGTTAGACTATGTTAAGAAACTAATACCTGAAACTGCAATGGTCAAAATT
TTGGAAAAATTATATGAAGAAGAGGTAGGAGTTGGGATGTATGTGTTGTA
CCCTTACGGTGGTATAATGGATGAGATTTCAGAATCAGCAATTCCATTCC
CTCATCGAGCTGGAATAATGTATGAACTTTGGTACACTGCTACCTGGGAG
AAGCAAGAAGATAACGAAAAGCATATAAACTGGGTTCGAAGTGTTTATAA
TTTCACAACGCCTTATGTGTCCCAAAATCCAAGATTGGCGTATCTCAATT
ATAGGGACCTTGATTTAGGAAAAACTAATCCTGAGAGTCCTAATAATTAC
ACACAAGCACGTATTTGGGGTGAAAAGTATTTTGGTAAAAATTTTAACAG
GTTAGTTAAGGTGAAAACCAAAGCTGATCCCAATAATTTTTTTAGAAACG
AACAAAGTATCCCACCTCTTCCACCGCATCATCAT

Interestingly the THC synthase gene has a CWCTTAGWC (Zhang et al. 2001, Proc Natl Acad Sci, USA, 98(22):12572-12577) motif at base 630. This is one base different from the motifs seen in different plants for mPIF integration (CWCTTAGWG) although Zhang et al report the outer base has only 61% conservation. Integration events mid gene (1635 bp full length) would be expected to multiply a truncated peptide but the active site including the FAD binding domain would remain un-altered at base 165.

### Homologs of the Cannabinoid Synthase Genes

The increased coverage of the THC synthase gene and its 90% homology to CBD synthase could be a result of many other novel synthase genes being collapsed in assembly.

### Terpene Biosynthesis

Terpenes are another class of molecules expressed in plants that exhibit antifungal, antibiotic and other medicinal properties like vitamin A and Taxol. Gallucci et al demonstrate the benefits of combination therapy of penicillin and various terpenes on MRSA. Vitis Vinifera or grapes have 40 unigenes related to the terpene synthesis (Martin et al., BMC Plant Biol, 10:226) and Cannabis has reports of at least 68 Terpenes using headspace gas chromatography and up to 140 terpenes (Ross and ElSohly 1996) consisting of approximately 90% monoterpenes and 7% sesquiterpenes and various other ketones and esters. One of the closest relatives to cannabis, Humulus lupulus or Hops has sequenced EST libraries extracted from the glandular trichomes (Wang et al. 2008, Plant Physiol, 148(3):1254-1266) identifying over 22 unigenes encoding terpene biosynthesis.

### Polymorphisms in the Human Endocannabinoid Pathways

To understand the variation found in the cannabinome and the impact of phytocannabinoids, the polymorphism in the human endocannabinoid pathways are of equal and relevant interest. Harismendy et al demonstrate SNPs which impact body mass index (BMI) in the Fatty Acid amide hydrolase (FAAH) and the monoglyceride lipase (MGLL) genes (Harismendy et al. Genome Biol, 11(11):R118). These genes encode enzymes that catabolize endocannabinoids, anandamide (AEA) and 2-arachidonyl glycerol (2-AG) respectively. The commonly used analgesic and thermoregulatory prodrug paracetamol is known to require FAAH to metabolize paracetamol with anandamide to form AM404. This metabolite is thought to be an endocannabinoid re-uptake inhibitor preventing anandamide clearance from the synaptic cleft analogous to SSRI drugs regulation of serotonin reuptake. This helps to explain one of the cannabinoids reported benefits in pain management (Hogestatt et al. 2005, J Biol Chem, 280(36):31405-31412). In addition, AM404 has been shown to be an agonist of the TRPV1 or vanilloid receptors much like capsaicin found in many cayenne and other red peppers and an inhibitor of cyclooxigenase COX-1 and COX-2. These findings prioritize a more thorough understanding of the 85 cannabinoids and the polymorphic diversity of the FAAH, MGLL, TRPV1 receptors and the genes encoding human cyclooxigenases.

The findings of Harismendy suggest that polymorphism content in the human endocannabinoid pathway can better guide patients to cultivars with more favorable cannabinoid content. Independent isolation of cannabinoids has resulted in FDA approved drugs (THC or Marinol™) but studies have shown a 330% increase in efficacy with combined CBD and THC delivery resulting in the European approved Sativex™ (Fairbairn and Pickens 1981, Br J Pharmacol, 72(3):401-409). Patients still report better outcomes from the whole plant extracts suggesting synergistic effects of the shotgun therapy and an interest in how each popular cultivar may vary in expression of active content. Cultivars that express THCV as another therapeutic cannabinoid are now being pursued. This genome sequence provides a tool to help selectively breed higher expression levels of various cryptic cannabinoids into plants to better study the impact of the cannabinoid and terpene repertoire.

### Description of ClustalW and Medicinal Genomics THC Synthase Sequences.

ClustalW is a tool which takes similar Sequences and “clusters” them together so one can see them aligned and compared to each other. As an example provided herein is a ClustalW of the 16 known THC Synthase sequences which were in Genbank to date.

Areas where polymorphisms existed were determined. Other Java based viewers can also be used. These can be very helpful tool for comparing new sequences and finding amino acid altering differences. This was done for multiple sequences from C. Indica genome which have some variation in the THC synthase DNA sequence and some of this sequence variance is Amino Acid altering making them very important variations as they impact the synthesis of THC and probably CBD and a variety of other Cannabinoids.

### Discussion

Gregor Mendel pioneered genetics working with Pisum sativum, an angiosperm with 10× larger genome and an 8× longer breeding cycle. The recently sequenced Date Palm genome highlighted the challenging genetics presented with a 7 year reproductive cycle (Al-Dous et al., Nat Biotechnol, 29(6):521-527). Cannabis cultivars flowers in 40-90 days making it an ideal candidate for genome directed selective breeding once many of the cannabis genomes are sequenced. Prior to this sequence dbEST, dbGSS, dbPLN, and dbHTG have a combined sequence for Cannabis of just over 2.05 Mb with 3944 entries. This study represents over a 65,000 fold increase in genomic data publically available for this plant and brings light to the polymorphism content and structure governing the medicinal synthase genes.

One of the challenges embarking on such a study is maintaining strong chain of custody of the plant matter to DNA as few countries have legal mechanisms to obtain plant material and legally sold cannabis has few quality and tracking standards to afford a properly designed genetic study. Material accessible through NIDA has been deemed less relevant as it fails to represent THC levels present in most strains used medicinally today.

As a result, the study described herein was aimed at sequencing one of the more popular C. sativa cultivar (“Chemdawg”) that has a controversial folklore over its origin to help drive a genetics based standard in the industry. Complementing this is the sequence of a triple back-crossed C. Indica strain (“L.A. Confidential”) where legal commercial entities are maintaining the seed line (DNA Genetics, Netherlands). This sequence can better aid the understanding of the genetics which govern cannabinoid expression and help build tracking and standardization tools to enable Cannabis extracts as a more measured therapeutic.

### EXAMPLE 2

### Methods

DNA was purified with Qiagen Mini and Maxi plant DNA purification Kits in Holland. Briefly, 500 mg of plant tissue was carefully diced with a razor and after addition of AP1 lysis solution homogenized with an IKA Turrax tissue homogenizer for 45 seconds on speed 10. Centrifugation steps were replaced with positive pressure filtration. Eluents from the final columns were re-purified with Ampure using a 1:1 volume of Ampure to sample (Beckman Genomics) and eluted from the magnetic particles with 65C ddH2O for 5 minutes. 10-20 ug of DNA (10-20 ng/ul) was delivered to Beckman Coulter Genomics and 454 Sequencing Service Center for library construction according to the manufacturers guidelines. 0.6% and 1.5% of the Sativa reads map to Chloroplast and mitochondrial genomes using Date Palm chloroplast as a reference and 47 mito plant sequences as a reference. Sativa cultivar “Chemdawg” and Indica cultivar “L.A. Confidential” were used as the first reference genomes (DNA Genetics only maintains LA confidential). CBD and THC levels are available at Full Spectrum labs (fullspectrumlabs.com). Sequencing of the Indica reference genome was accomplished with sixteen 454 GS FLX+ 700 bp runs delivering and 14× coverage. Genome sequencing and assembly was performed by the 454 Sequencing Service Center in Branford CT assembled with Newbler. The Sativa strain was sequenced to 327× coverage with 2×100 ILMN HiSeq (651M reads, 131 Gb of PF filtered data) sequencing reads performed by Beckman Genomics The Illumina and 454 assemblies 10,11, & 12 were assembled with CLCbio Genomics Workbench 4.7.1. SNP calling was performed with CLCbio Genomics Workbench 4.7.2. For Illumina data a minimum of 2 pairs was required to call a SNP and the default Neighborhood Quality Scores (NQS) were used. SNP lists were exported as csv files and compared with perl scripts for overlapping coordinates.

### Results

The outbred Sativa cultivar Chemdawg or “CD Sativa” was sequenced to over 320× coverage with Illumina 2×100 paired end reads. Single lane assemblies and multi-lane assemblies produced very similar fragmented assemblies and demonstrated both high AT content (65.6%) and a high polymorphism rate (0.5% intra-cultivar, 0.63% intercultivar. To address the polymorphism rate in the genome, a triple backcrossed pure Indica cultivar named LA Confidential or “LAC Indica” (DNA Genetics, NL) was chosen to build a high-quality reference genome with over 19.5 million 454/Roche GS FLX+System 700 bp reads. The Indica genome was assembled with three different alignment stringencies on CLCbio workbench and Newbler. Genome assembly size estimates of 286-340 Mb for the CD Sativa cultivar were obtained based upon the Illumina-CLC assembly, and 676-727 Mb for the 454 LAC Indica cultivar based upon the 454 sequencing assembly with N50s of 2.6 Kb. The variation in genome size estimations are a result of the high polymorphism rate in the genome collapsing, or occasionally splitting, the maternal and paternal alleles in assembly, and is a known challenge with modern DNA assemblers. Therefore, the CD Sativa assembly is likely smaller as a result of shorter reads inability to phase highly polymorphic branch points in the assembly despite the 20 fold higher coverage. The LAC Indica results are supported by van Bakel's genome assembly size estimates for Purple Kush (PK Indica) and flow sorting experiments suggesting 1.4 pg per diploid genome (Sakamoto).

To assess genome completeness, all cannabis DNA sequences in genbank were aligned to the Indica reference and significant blast hits for over 98.3% of the entries were found. An RNA-Seq assembly is publically available (medicinalplantgenomics.msu.edu) for a different Sativa cultivar (“Mexican or CSA”), and BLAST results confirmed that over 89% and 85% of the 69,557 transcripts from the CSA cultivar were present in the LAC Indica reference (Any E score, E score <E-10).

Most of these CSA entries were mRNA sequences and thus enriched for euchromatic sequence. To assess the heterochromatic coverage the number of reads not mapped in the varying assemblies was measured (filtered of dots and polyclonals). These ranged from 10% of the reads at the default alignment stringency (0.8) to 33% of the reads at the most stringent mapping conditions for the LAC Indica data. Comparisons to the recently published PK Indica genome assembly indicated that the LAC Indica genome assembly from Newbler is likely the most accurate genome estimate, while the CD Sativa assembly represents the less repetitive portions of the genome addressable with short read sequencers. When all of the 19.5M LAC reads were mapped to the PK Indica Cansat3 assembly 3.7M reads did not map (by comparison, all LAC Indica reads mapped back to the LAC Indica reference created 1.64M reads which did not map) and 15.8 Mbp of PK Indica contigs had zero coverage. Assembling these un-mapped reads produced 140,660 contigs larger than 500 bp. Only 10,394 of these mapped to the PK Indica Cansat3 transcriptome, leaving 130,266 unique contigs comprising 79 Mb of sequence unique to LAC Indica. 31% of these contigs had Blast hits for arabidopsis thaliana at an 0.01 E value cut off.

### Polymorphisms

To assess polymorphisms on a draft genome, reads were remapped to the consensus CLC assemblies to look for SNVs. This produced predominantly heterozygous SNVs for selfmappings, but heterozygous and homozygous SNVs for cross cultivar mappings with a Ti/Tv of 1.62-1.84. As expected, the outbred CD Sativa cultivar had more variation than the triple backcrossed LAC Indica, with both cultivars exhibiting a high degree of polymorphism as compared to the variation content seen across the human genome or Arabidopsis genomes. The larger Newbler LAC Indica assembly of 676 Mb (676 Mb contigs >500 bp, 727 Mb all contigs) discovered 925,602 SNVs with a Ti/TV 1.71 and a SNV rate closer to 0.13%. All of the CD Sativa and LAC Indica reads were then mapped to PK Indica and 4.5M and 3.8M SNVs, respectively, were found. Of these SNVs, 397,754 were shared (42% and 26%) between LAC Indica and CD Sativa and 1.23M were shared (32% and 27%) between LAC Indica/CD Sativa & PK Indica implying high diversity amongst the Cannabis cultivars, with a closer relatedness of PK Indica to LAC Indica.

### Synthase Genes

The THCA synthase genes display an increased polymorphism rate next to the genome at large (˜2% vs 0.6%), likely explained by this being a gene governing the dominant phenotype selected for with recreational breeding. Increased polymorphism rates can also be associated with collapsed copy number variations. In preliminary assemblies, read coverage indicate that the gene family has gone through several duplication events as described previously. Evidence for a gene expansion could also be seen in LAC Indica and CD Sativa with the increased genome coverage in this location compared to the genome average. One can also see more phased alleles than expected with a diploid plant. Both LAC Indica and CD Sativa cultivars exhibited six fold higher coverage in these regions. Increasing the coverage with the Newbler LAC Indica assembly broke these polyallelic contigs into different haplotypic contigs affording better amino acid prediction. Although it is tempting to assume this gene expansion explains the reported increased THC content in these cultivars, one must minimally demonstrate the gene expansions are transcriptionally active, in frame and not mis-sense mutated pseudo genes. As a result, segregation of the haplotypes in assembly is imperative in making use of RNA-Seq data in order to assess if any of these genes are expressed in frame. Subsequently one can stratify the RNA-seq mappings in an allele specific manner across the various tissues.

In this regard, others report convincing data in regards to the expression of transcription factors and their potential role in hemp to PK Indica differences. Likewise they also suggest the observed AAE3 copy number variation being more important to increased cannabinoid content than THCA & CBDA synthase gene expansions stating “Our analysis indicates that amplification of cannabinoid pathway genes does not appear to play a causative role in this increased expression”. The AAE3 copy number increase is interesting and could explain higher levels of cannabinoid precursor, yet higher chemical diversity of cannabinoids is expected to happen downstream of CBGA formation as most cannabinoids can be folded from this substrate or its propyl “varin” counterpart (de Meijer, 2003, Genetics, 163(1):335-346).

However, even with this increased copy number of AAE3, there does not appear to be a large difference in expression of this gene in Finola (hemp) compared to PK Indica (marijuana). Likewise, Finola is not a high CBDA cultivar and better classified as a THCA loss of function mutant with a functional CBDA synthase gene, which affords slightly higher (<%2) CBD expression since the CBGA competitive THCA synthase is dysfunctional. As a result, a simple point mutation as described by Kojoma et al. could more easily explain differences in Finola to PK and one might not expect to see a change in genomic architecture to simply reduce THCA synthase activity. Higher CBDA cultivars like Cannatonic are likely to provide more clarity on the effect of copy number on AAE3 expression.

Unlike AAE3, the THCA synthase and CBDA synthase genes showed differential expression in Finola vs PK Indica, despite their copy numbers being similarly expanded from Finola to PK Indica. Increased copy number and increased expression do not always deliver increased peptide activity. In the case of the gene expansion in LAC Indica this is partially due to missense or nonsense SNVs in or just downstream of the FAD binding domain of the expanded THCA and CBDA synthase sequences. As a result, the copy number expansions need to be scrutinized in regards to their transcriptional activity and the translational products the variants encode. To complement the sequence provided by van Bakel where they state ‘on the basis of our inability to assemble these into functional protein-coding genes, we conclude that the THCAS reads in ‘Finola’ and CBDAS reads in PK are likely to be caused by the presence of pseudogenic copies', the analysis herein was focused on the long reads to help phase these polymorphic gene families.

Phased sequence from long reads is essential in determining the translational code of such highly polymorphic assemblies. Even C terminal in frame truncated synthase genes exhibiting RNA-Seq expression and containing an intact FAD binding domain (N terminal) need to be taken into consideration as potential cannabinoid synthase genes, as opposed to assuming them to be pseudo genes.

In this regard, the LAC Indica assembly herein had four full length contigs (#20041, #32071, #34396, #20817) with homology to THCA and CBDA synthases and 10 partially homologous contigs with truncated ORFs. The full length contig, in particular, #34396, 81% sequence similarity to both, was highly expressed in the PK Indica RNA-Seq data but was absent from the PK Indica Cansat3 genomic assembly. In fact, the PK Indica Cansat3 genomic assembly only had one THCA synthase gene (PKcontig#19603) in the genome browser and the reported “THCAS like” sequences could be deduced via comparative alignment with LAC Indica. Failure to split these contigs can negatively effect resequencing alignments to this reference collapsing the entire gene family into highly covered and divergent loci. In addition, many of the PK homologs (PK_{—}20093.1 & PK_{—}09375.1 and PK_{—}23203.1) are truncated on the 5′ end and missing start codons. Confirmation of the THCAS-like sequences also revealed more full length THCAS-like sequence in LAC Indica where Cansat3 scaffold 49212 coded for a truncated peptide. The PK RNAseq data (SRR352202) supports an extended 5′ end but 5′ sequence bias creates a truncated peptide with an alternate start codon for transcript PK 09375.1.

Nevertheless, evidence for fully functional THCAS-like sequences exist in LAC Indica but a comparison to CD Sativa shows two of these genes to have broken open reading frames and two of them to appear functional. Sativa's were traditionally bred for long fiber stalks and later crossed with Indica's to acquire their pharmaceutical phenotypes and are known to express different chemotypes.

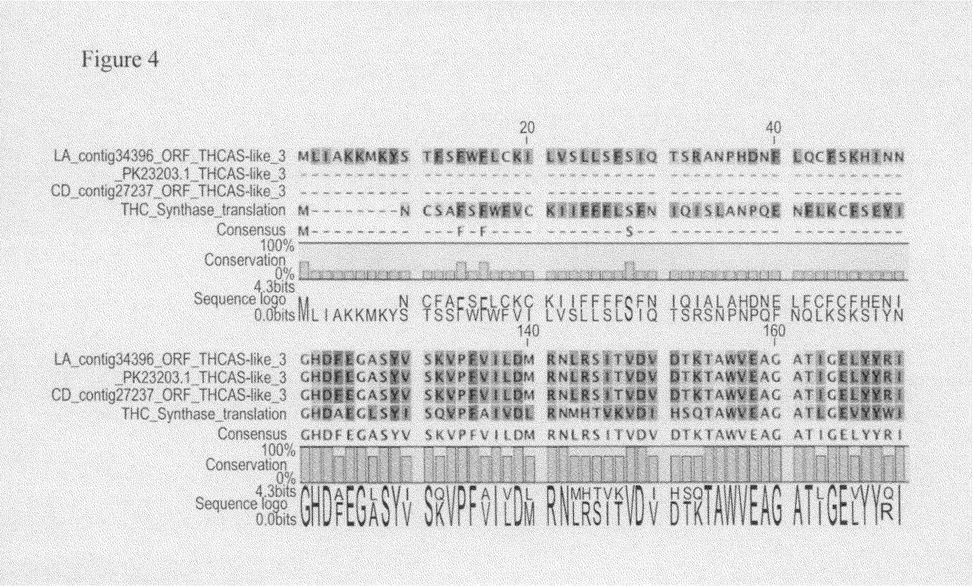

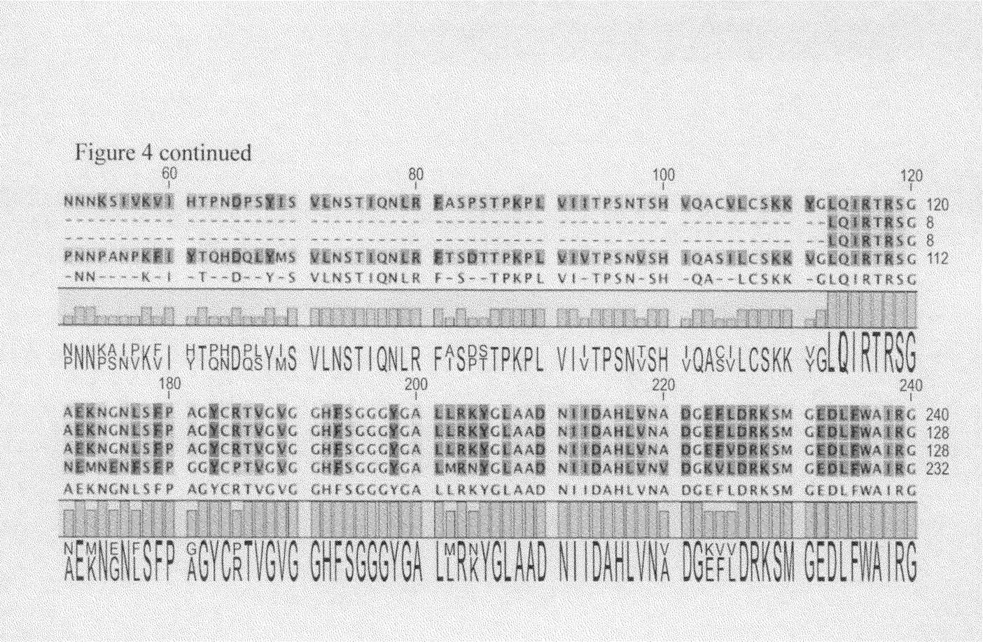

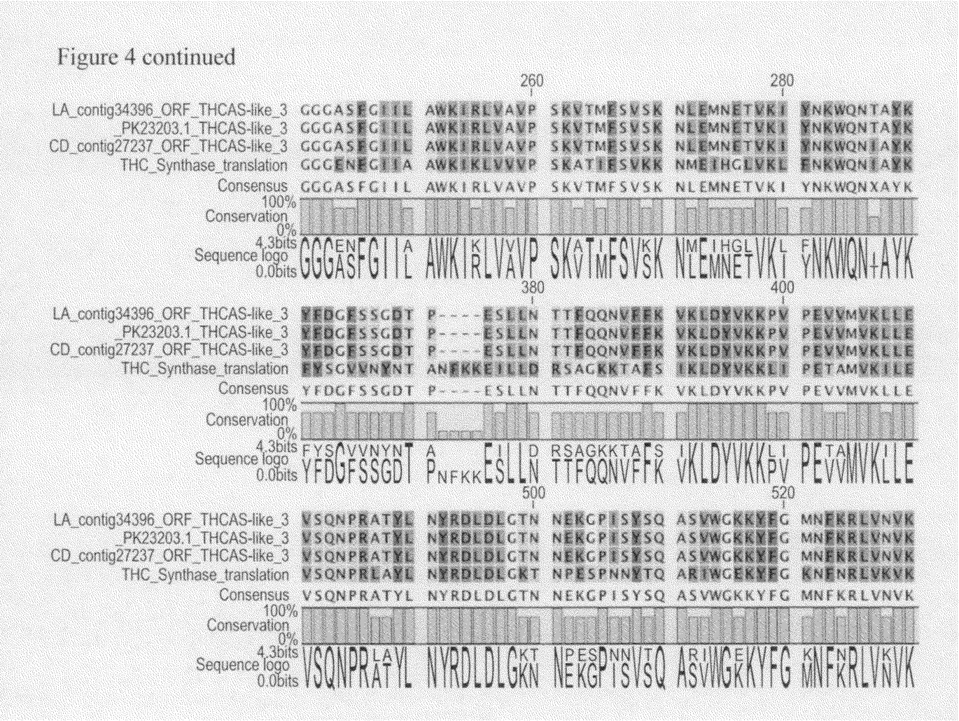

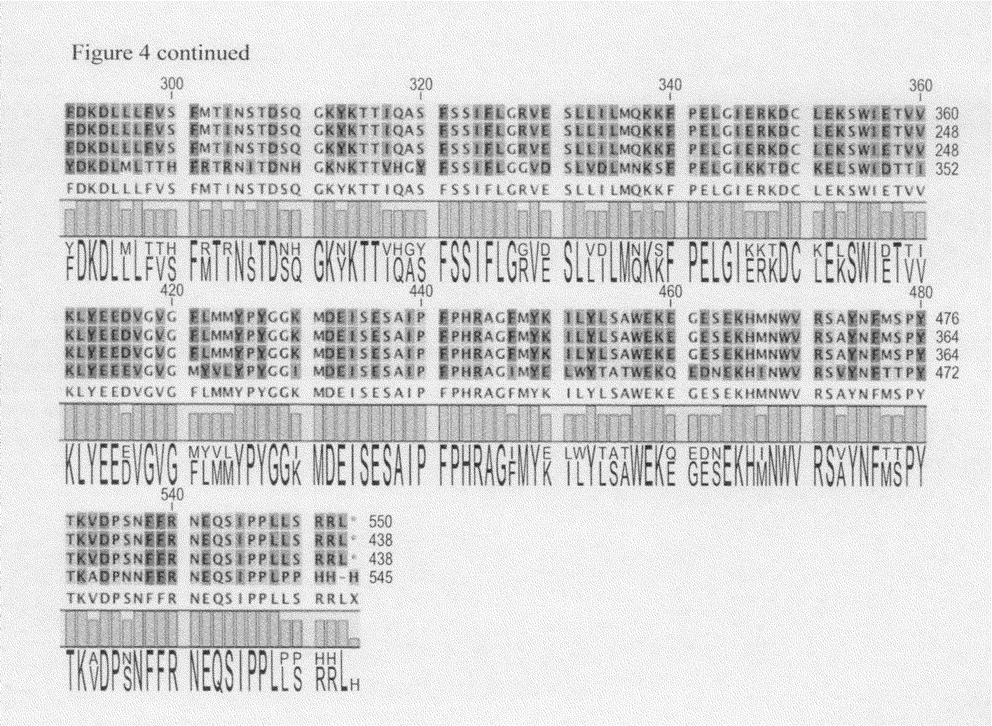

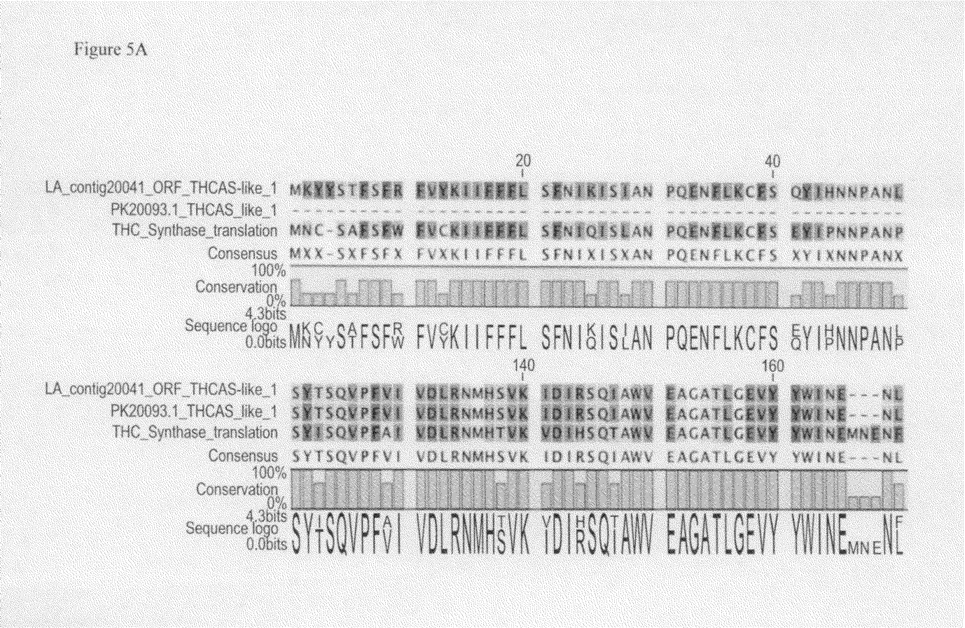

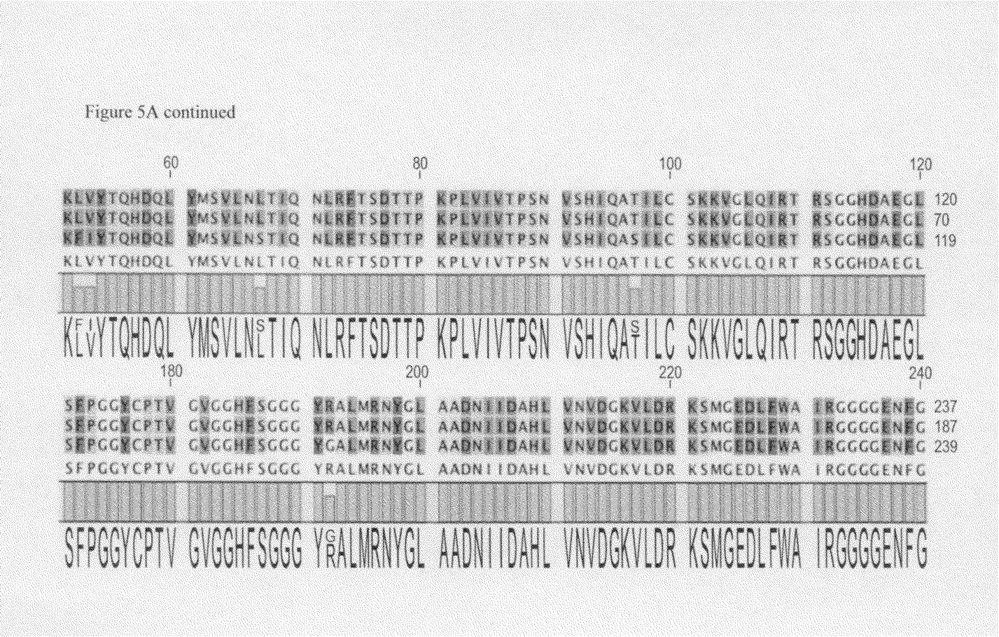

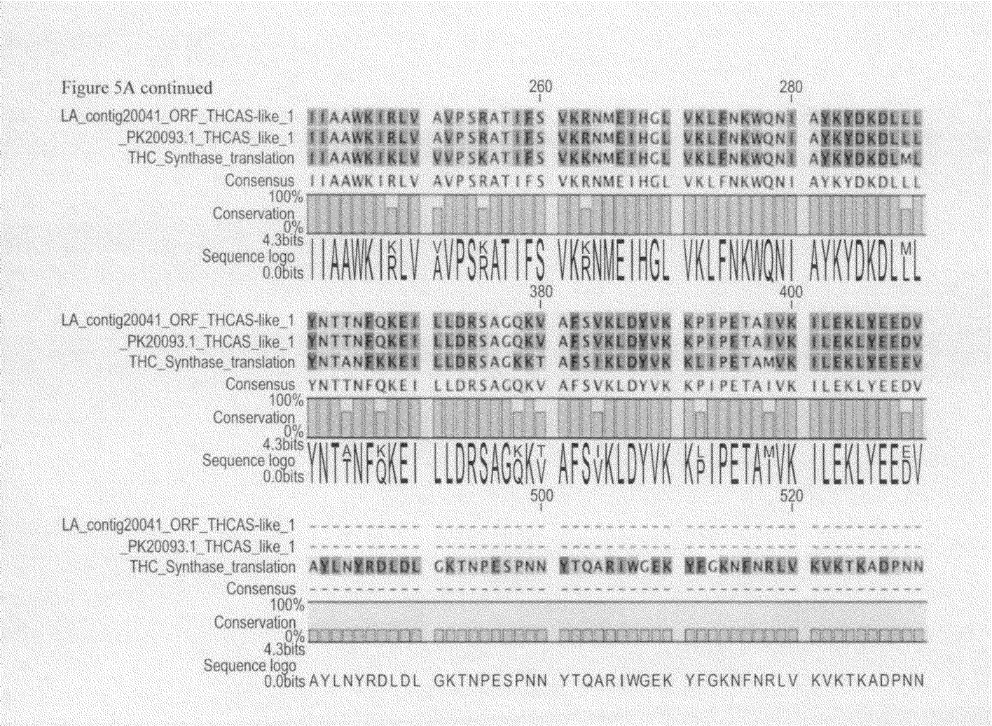

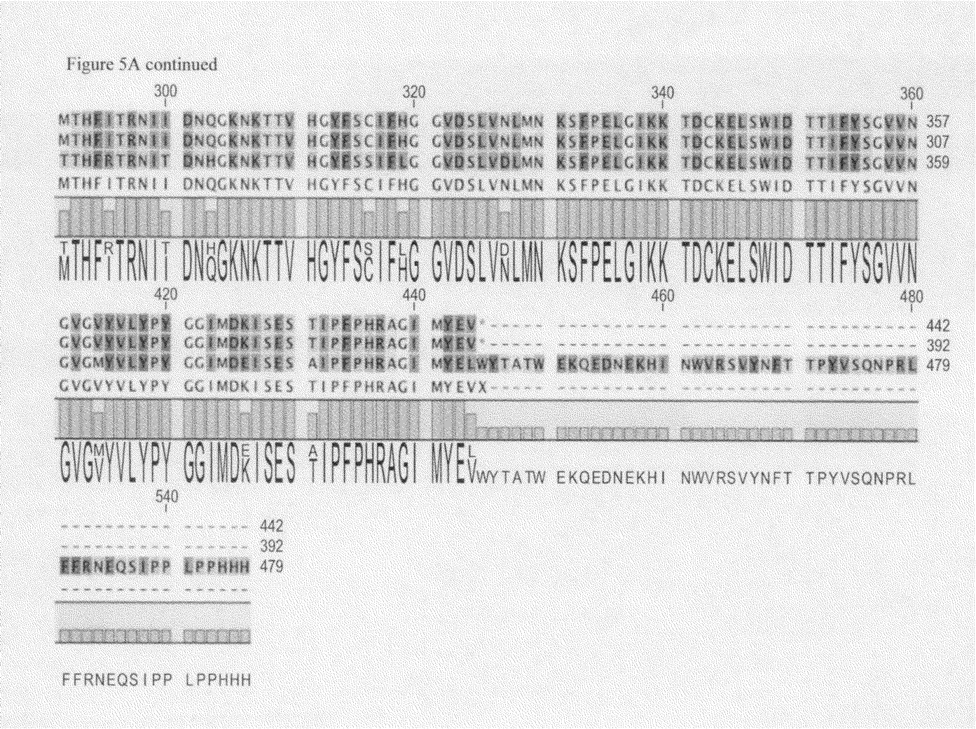

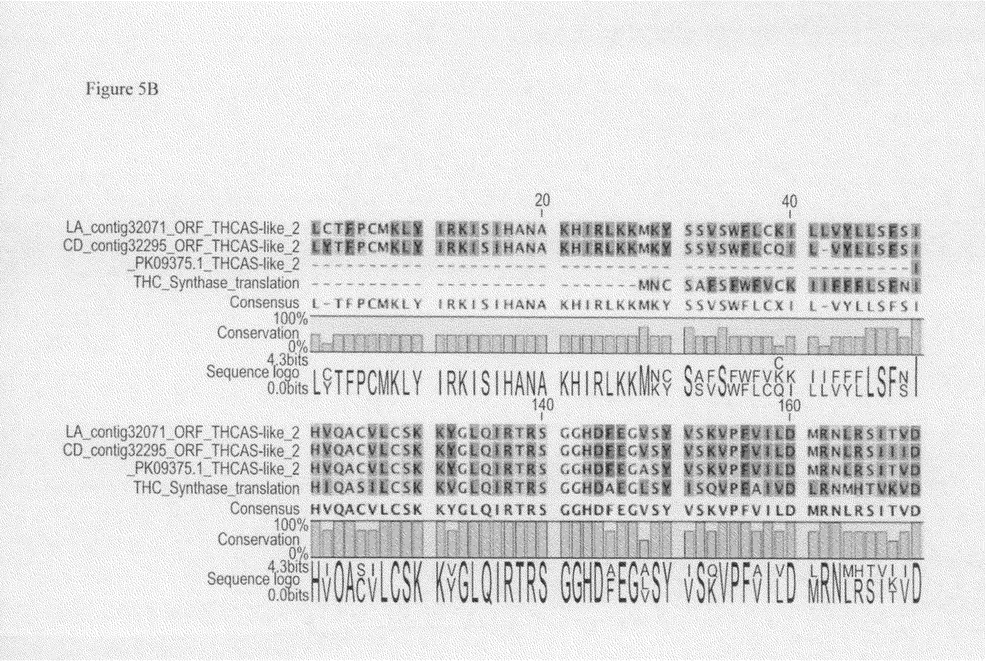

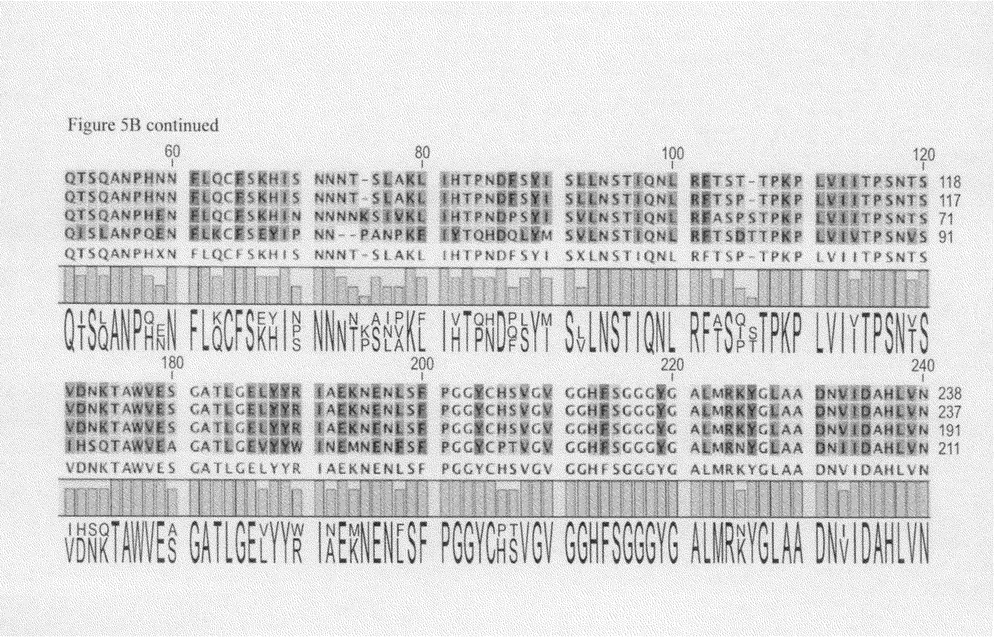

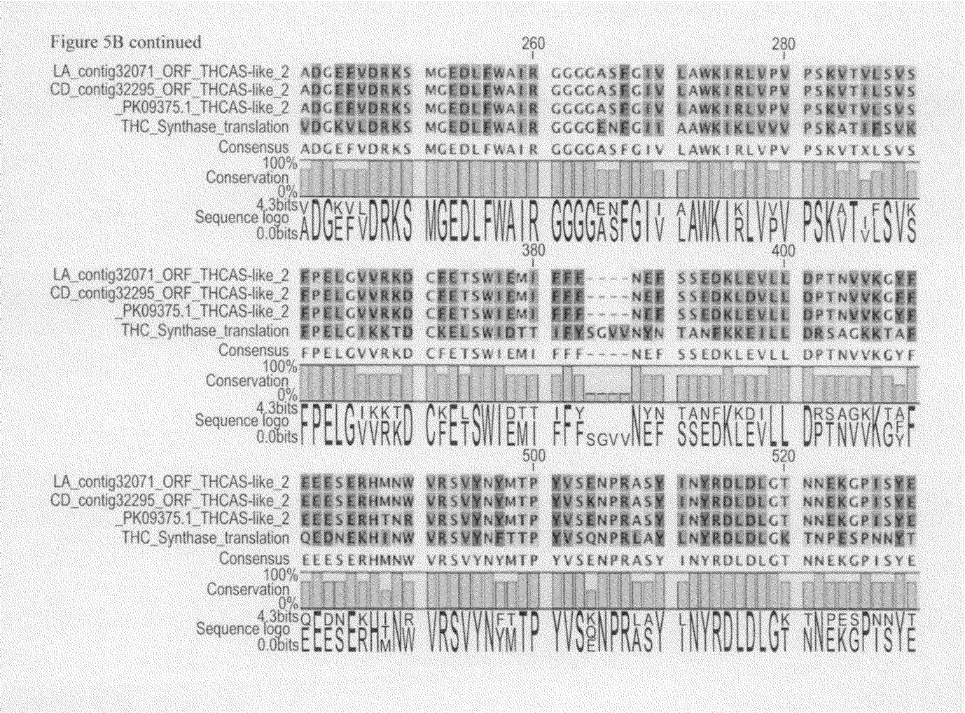

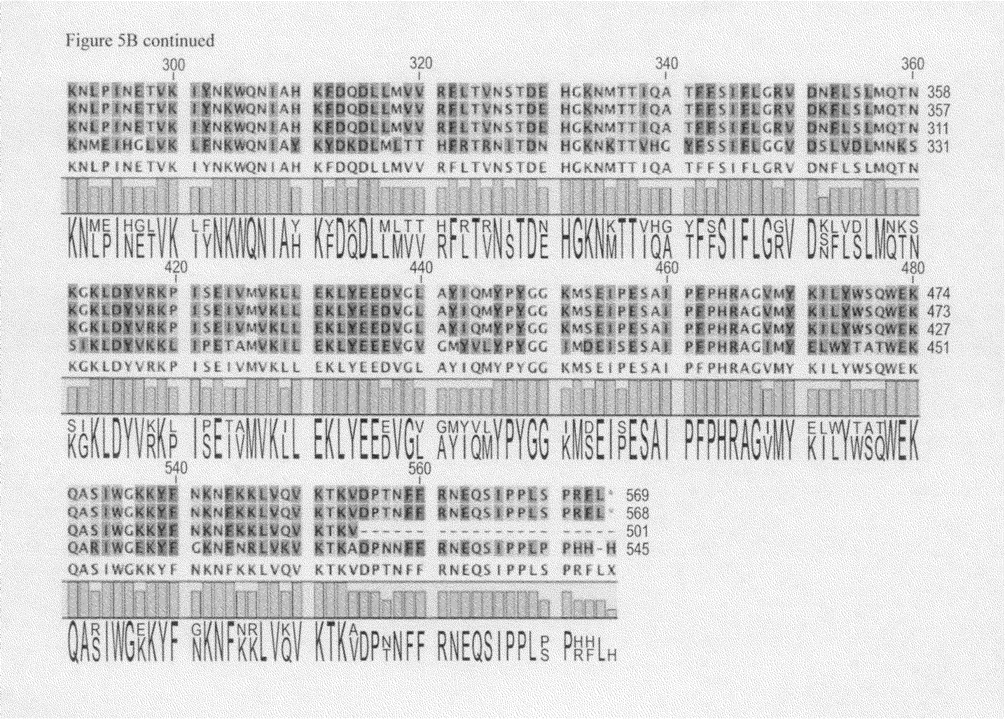

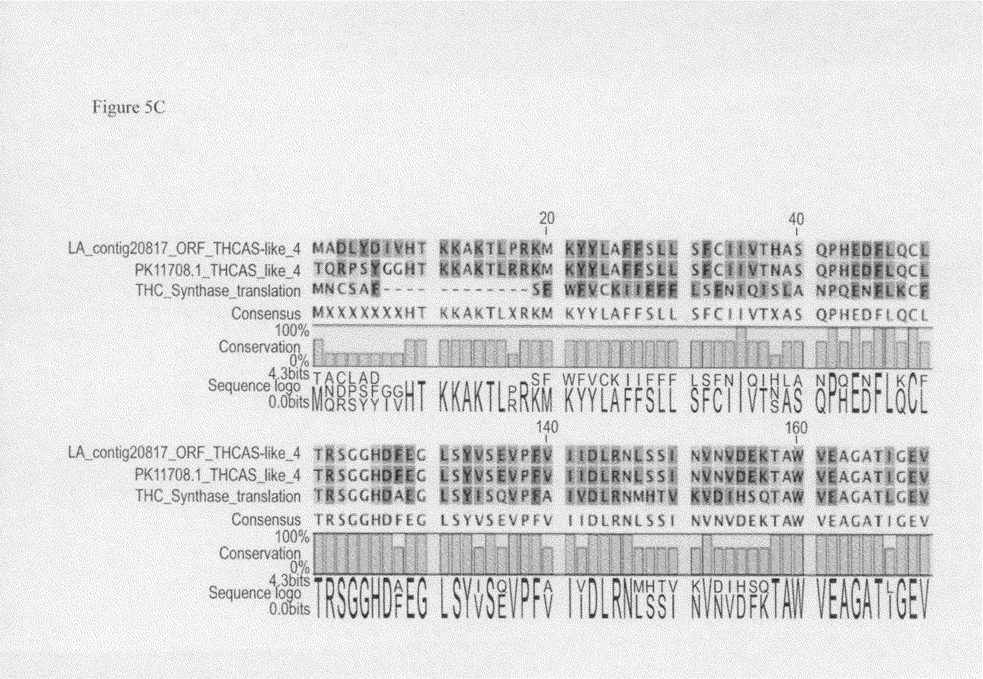

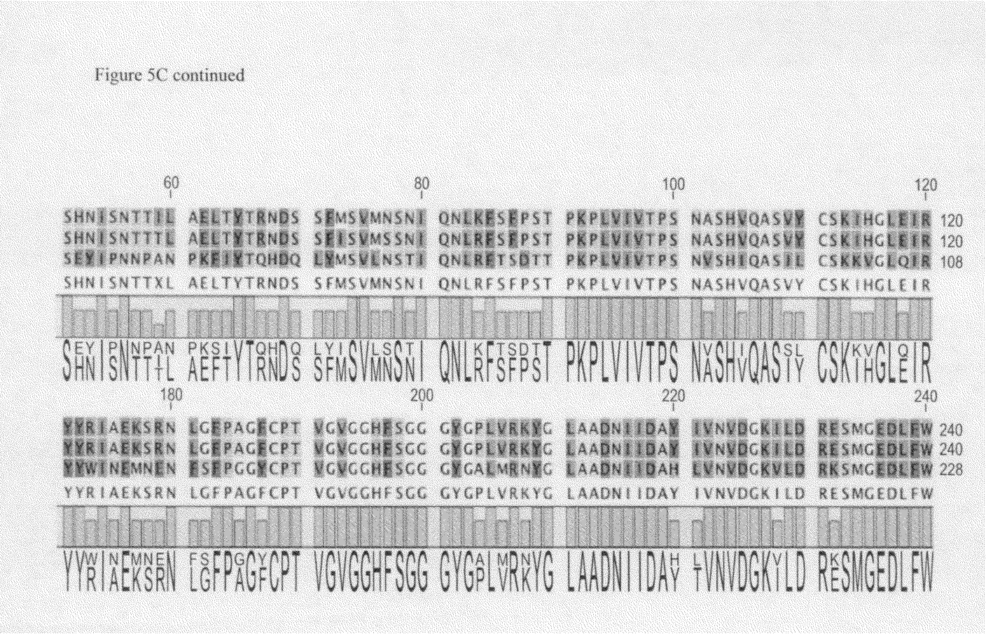

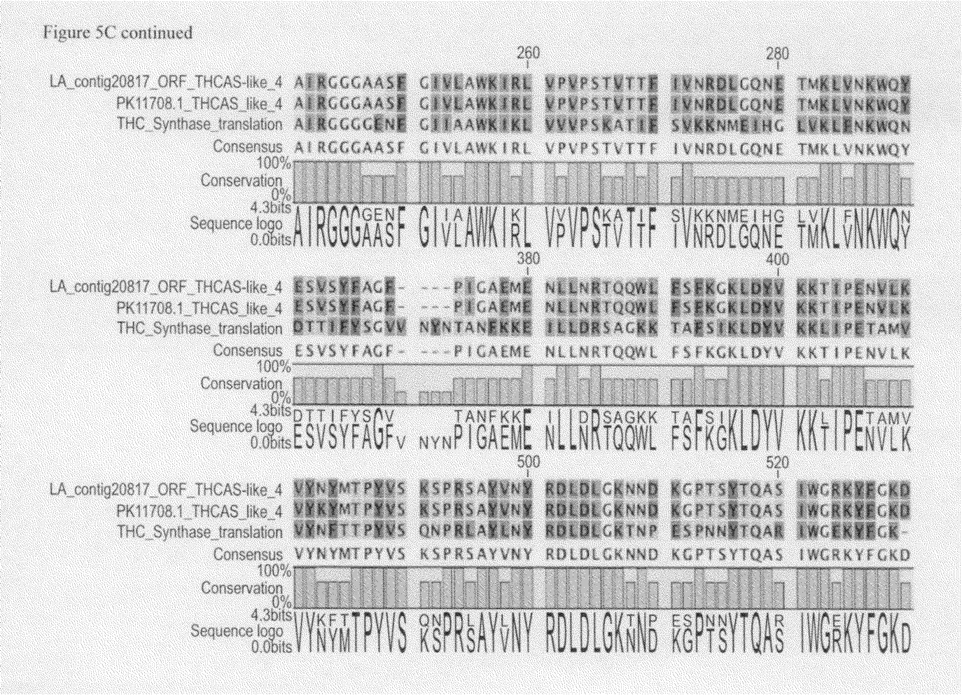

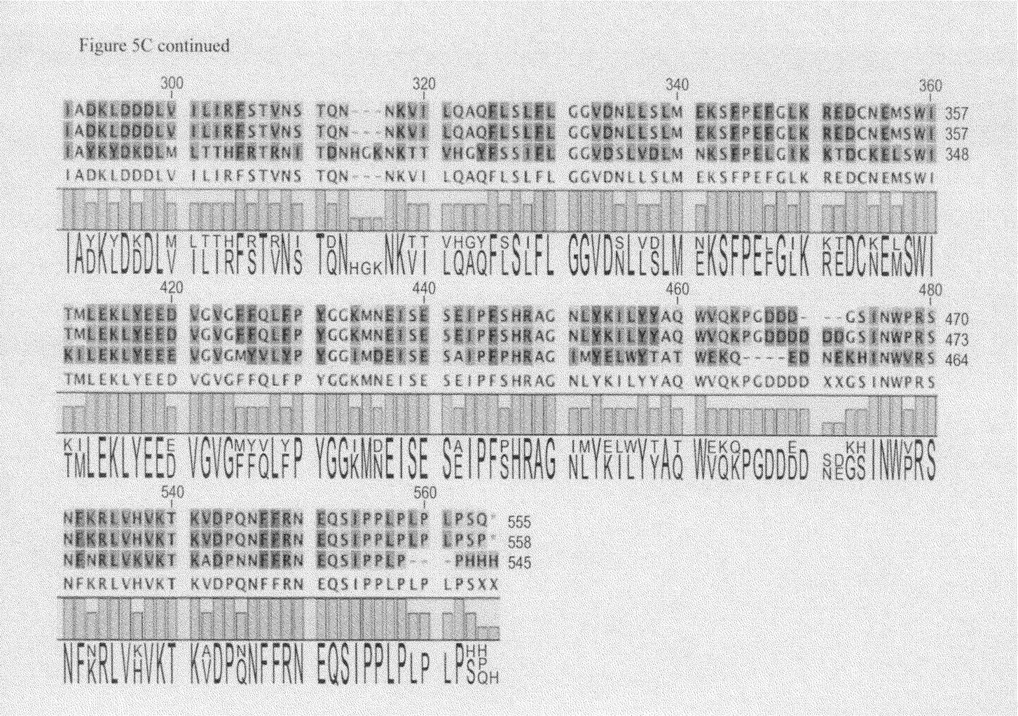

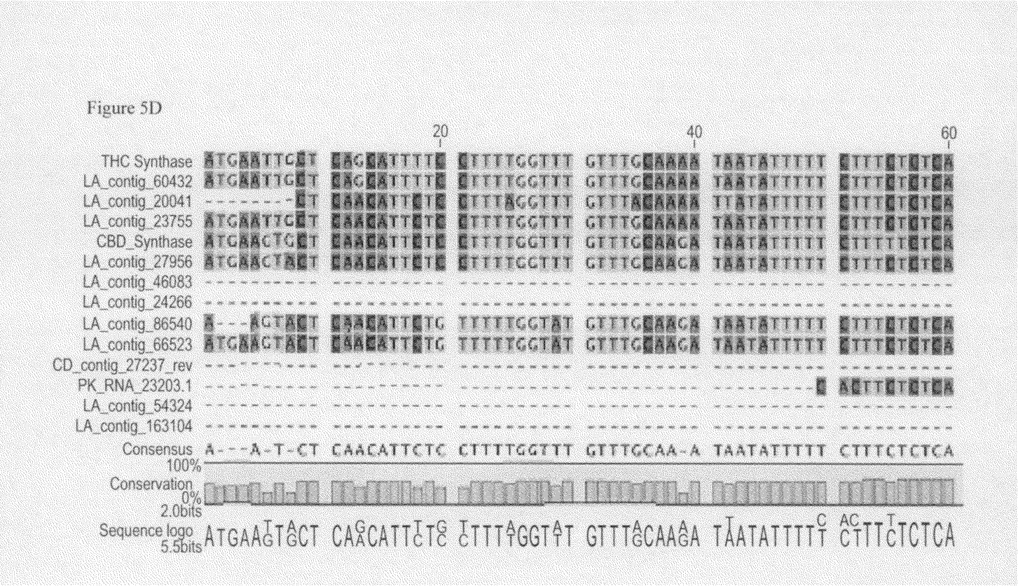

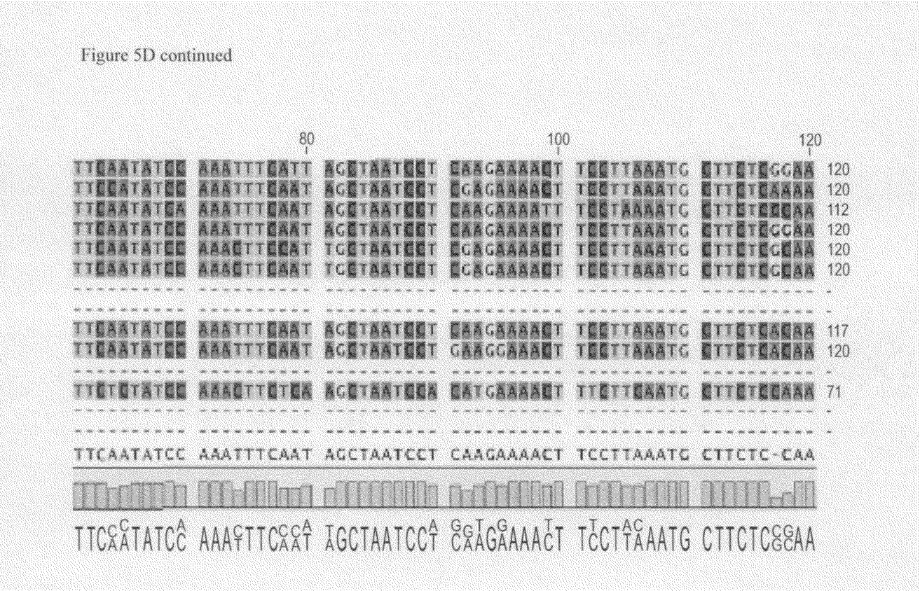

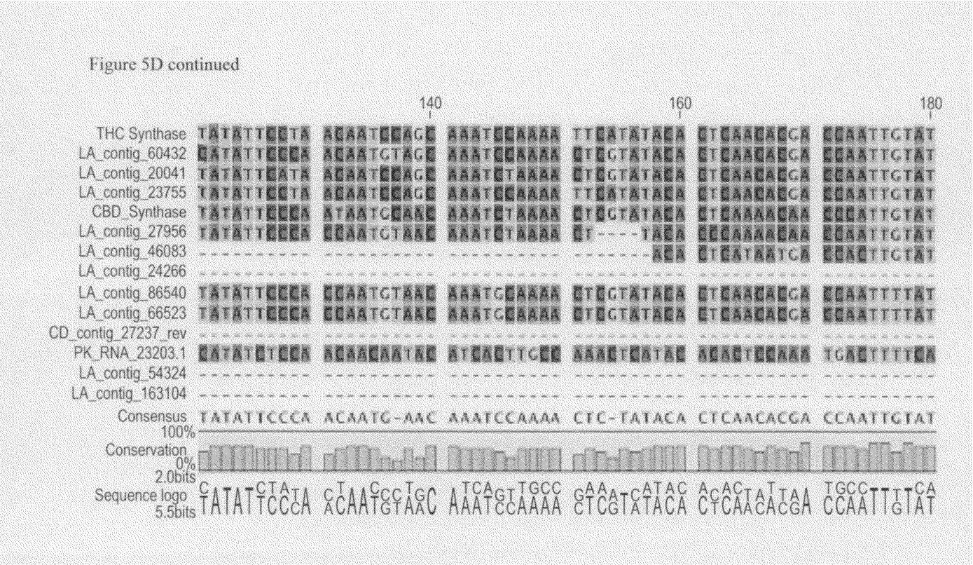

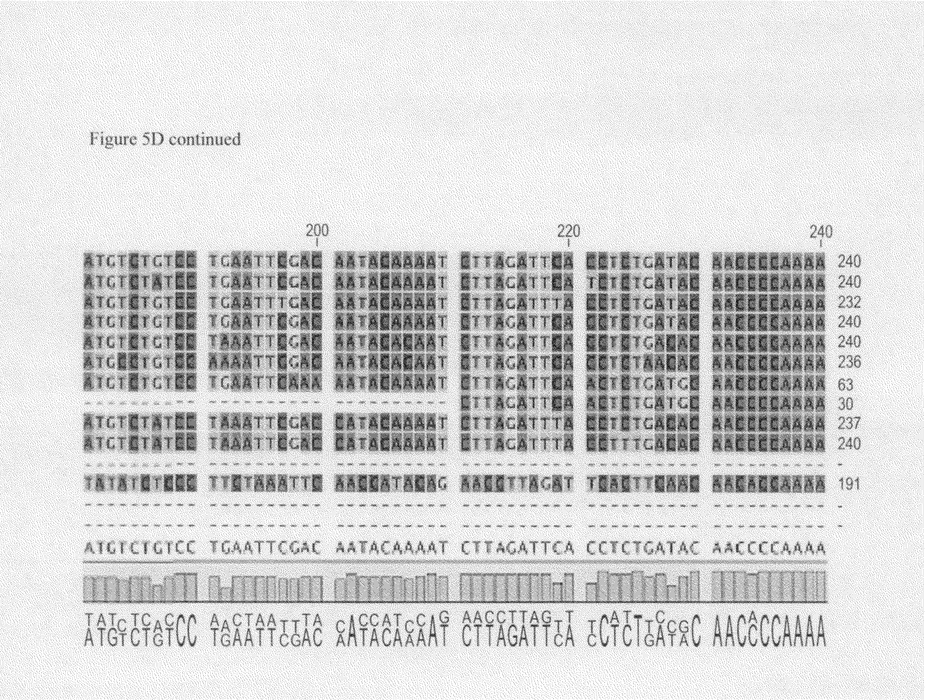

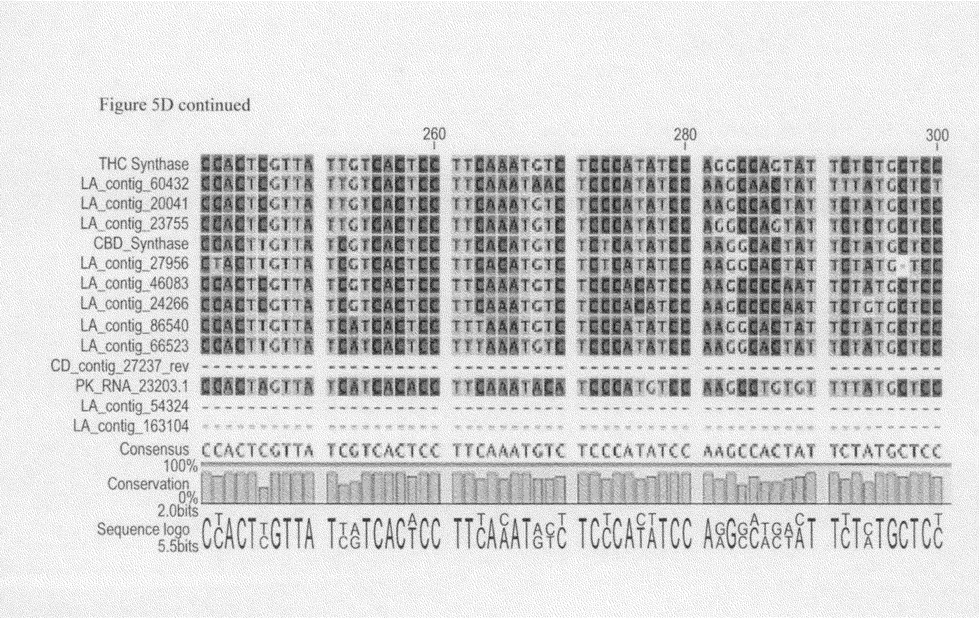

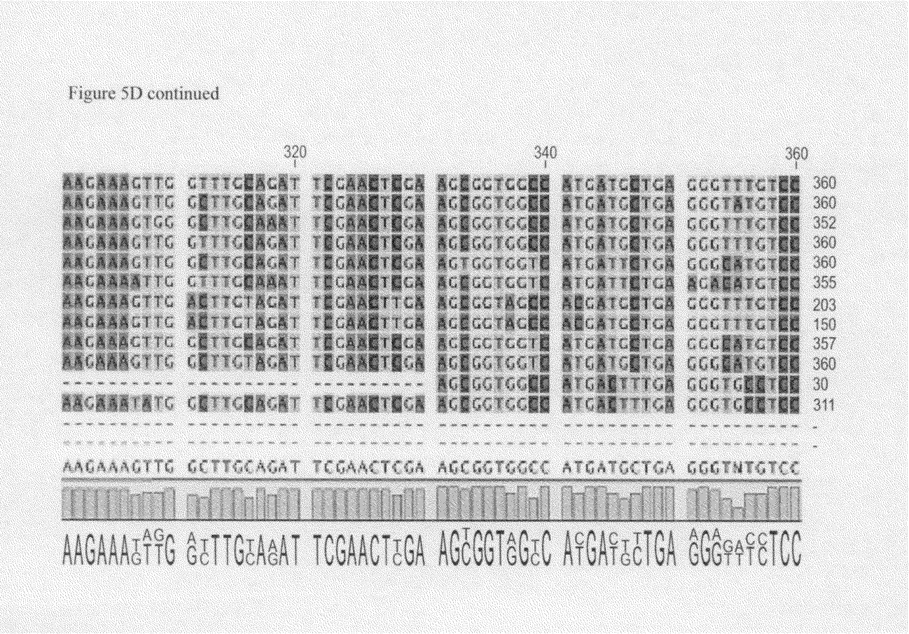

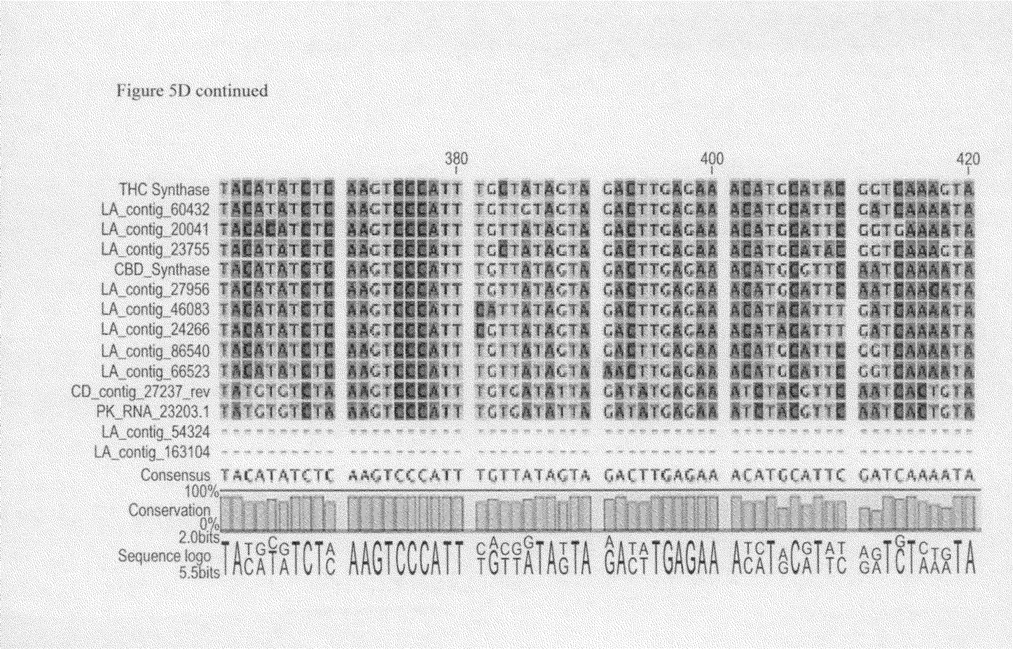

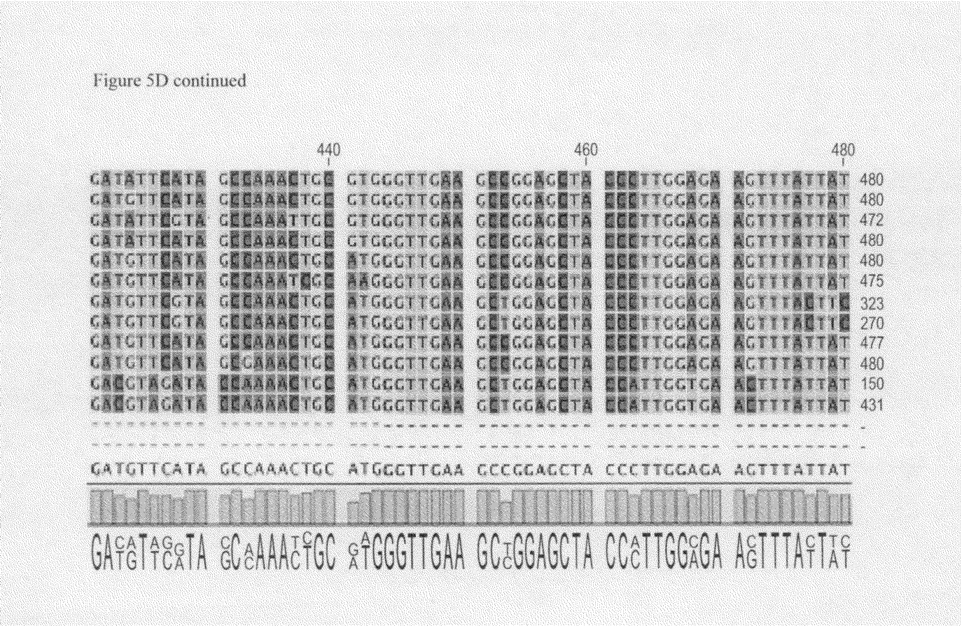

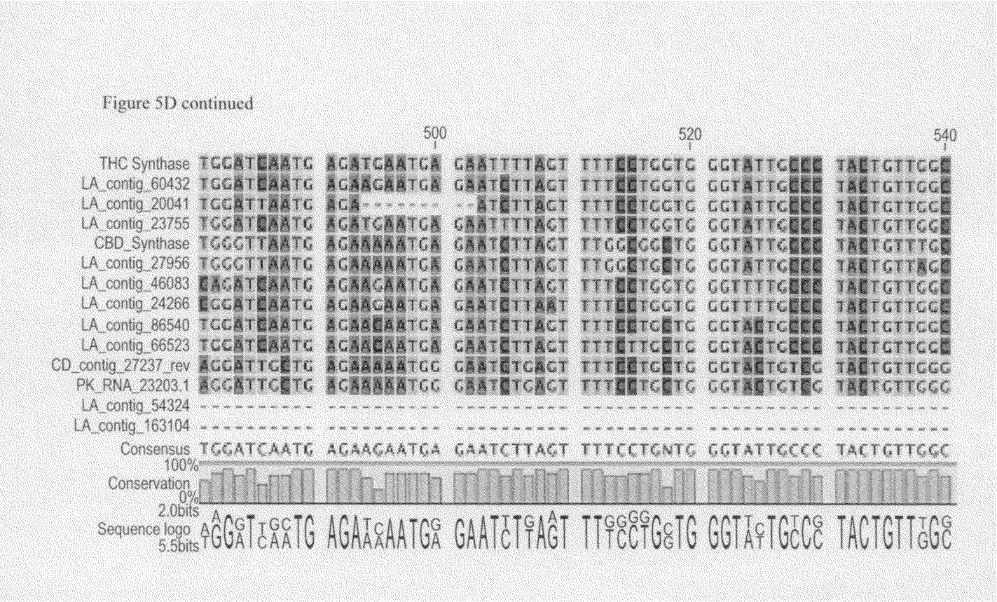

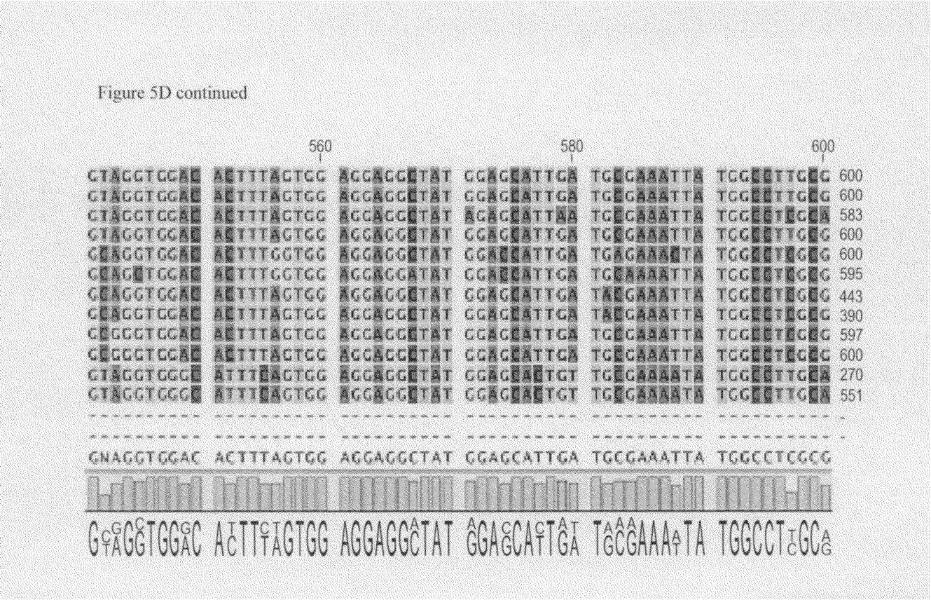

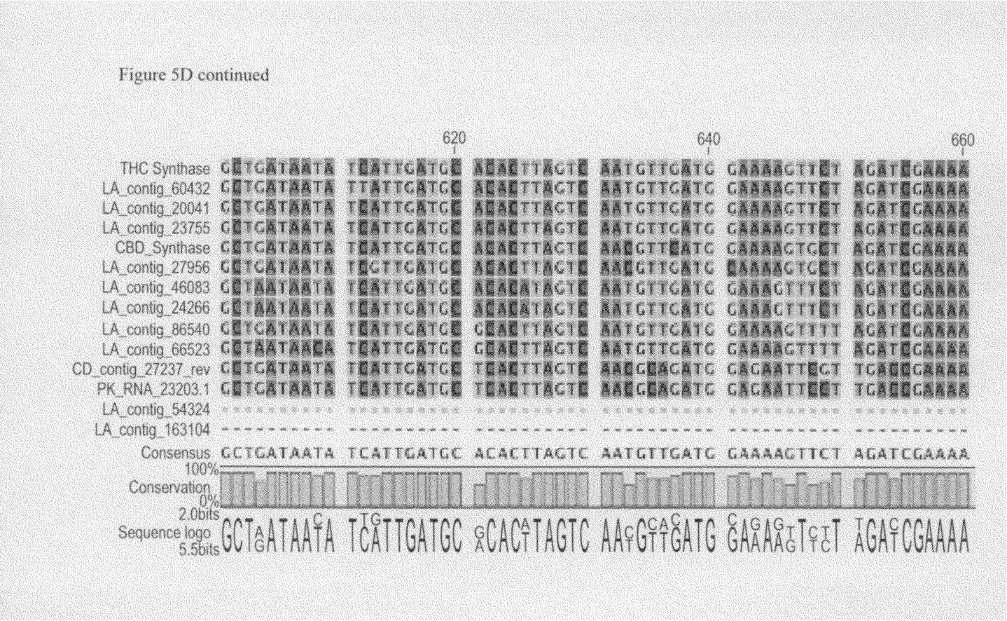

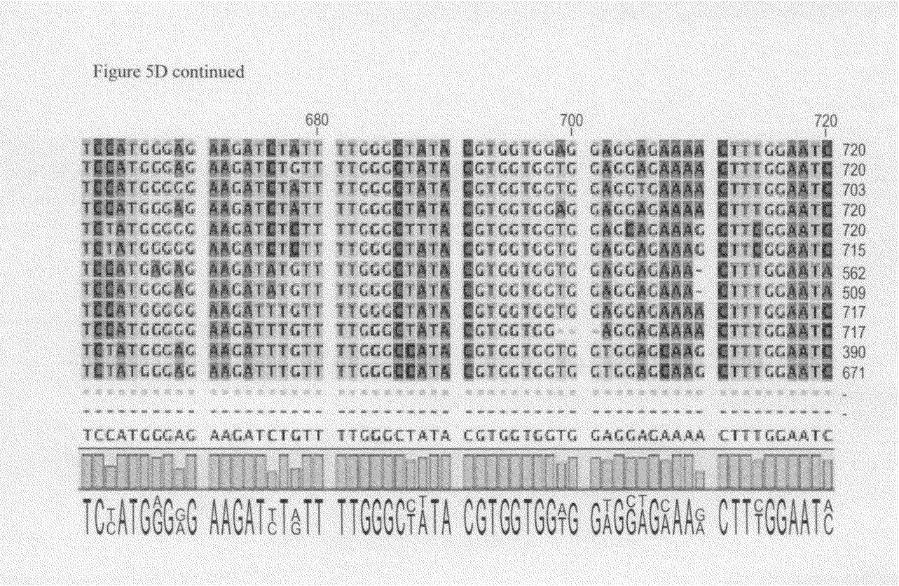

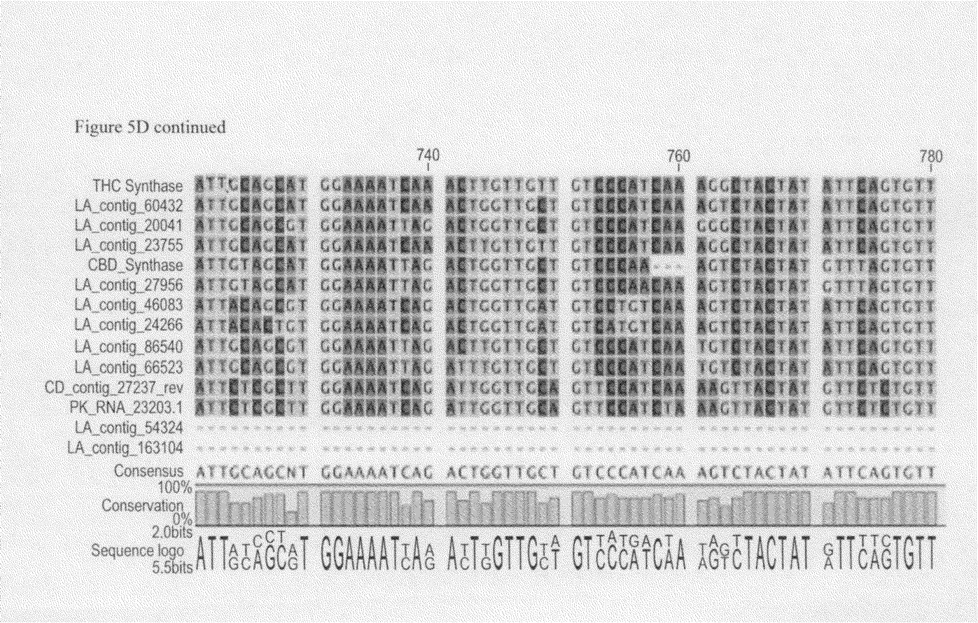

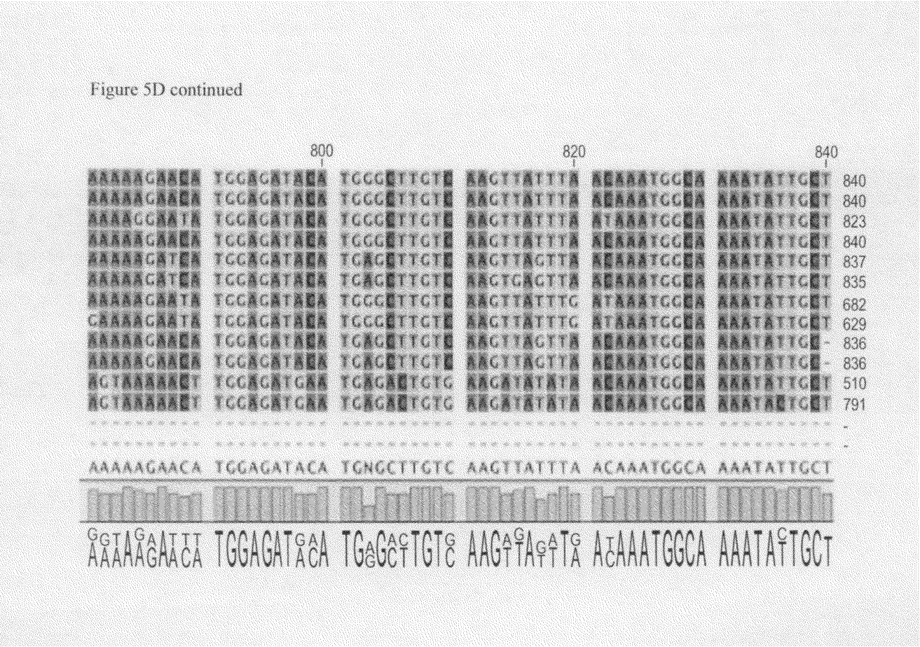

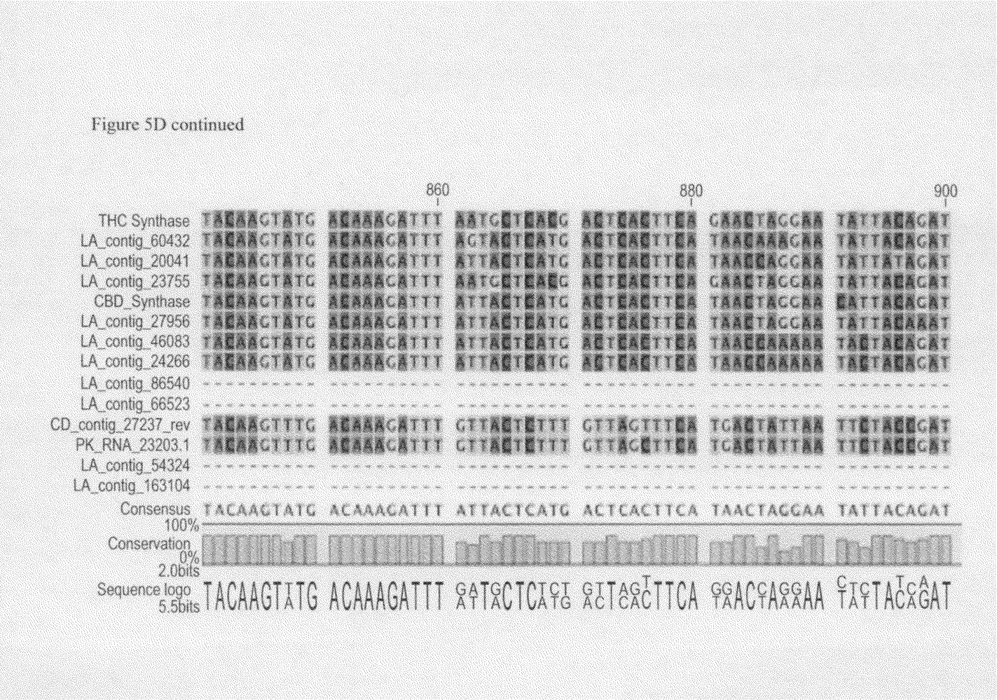

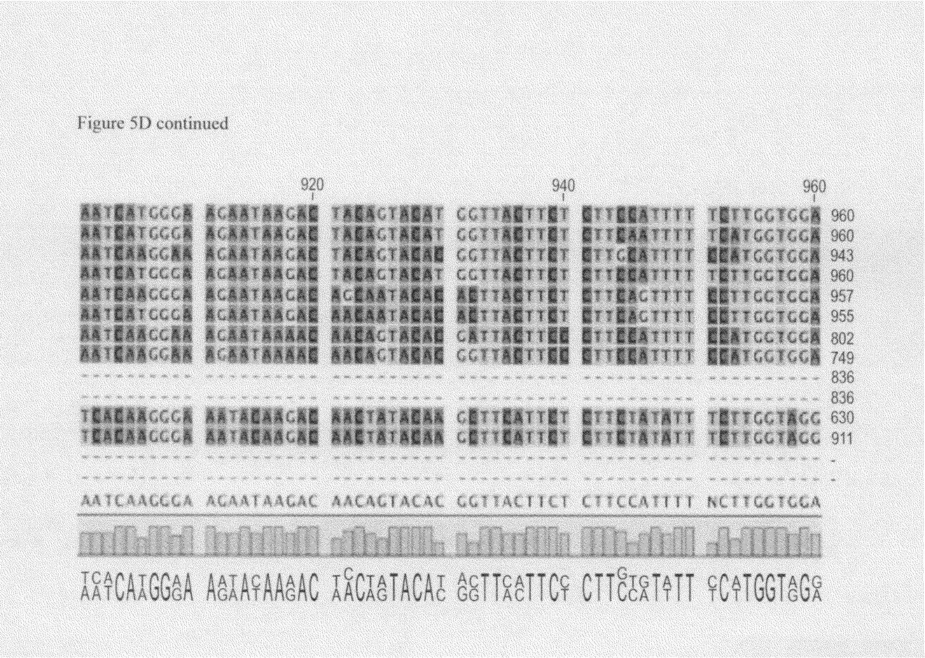

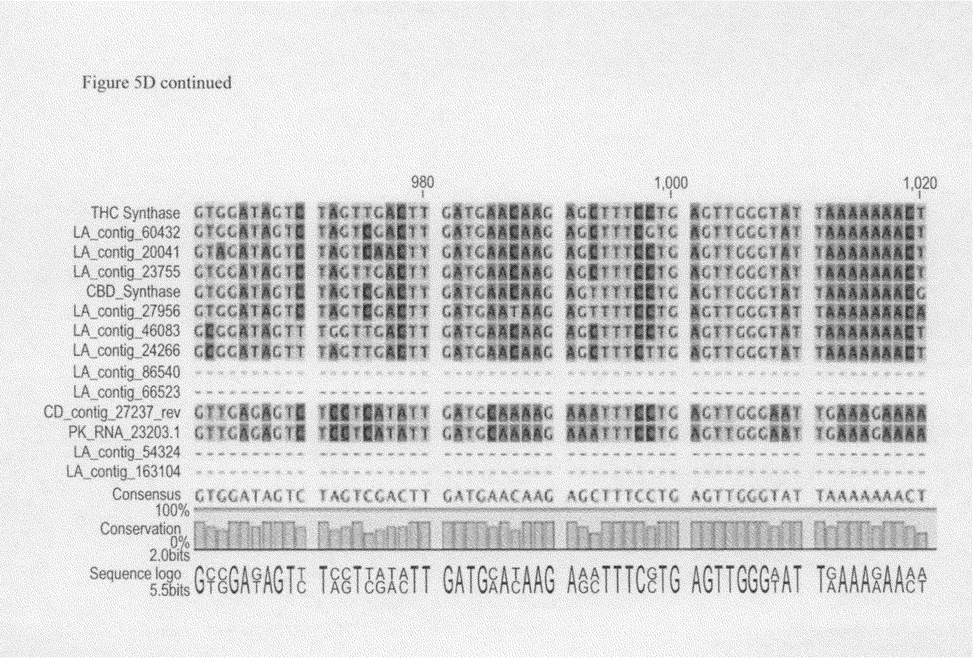

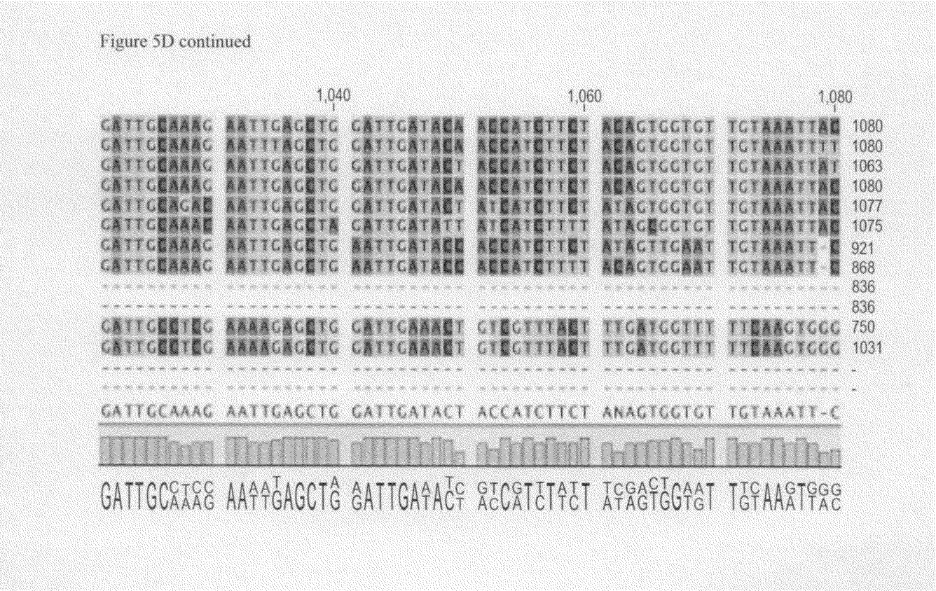

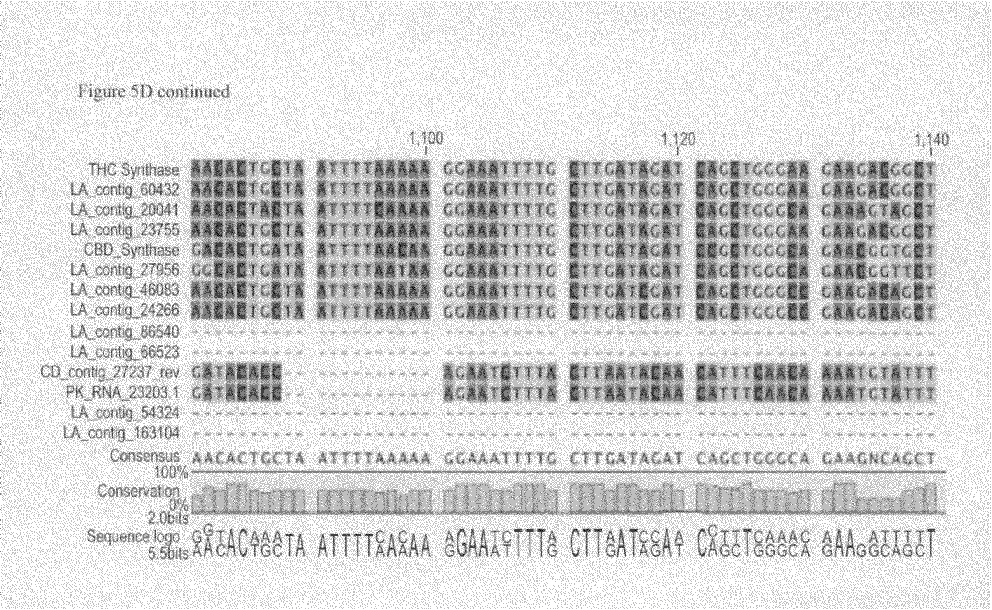

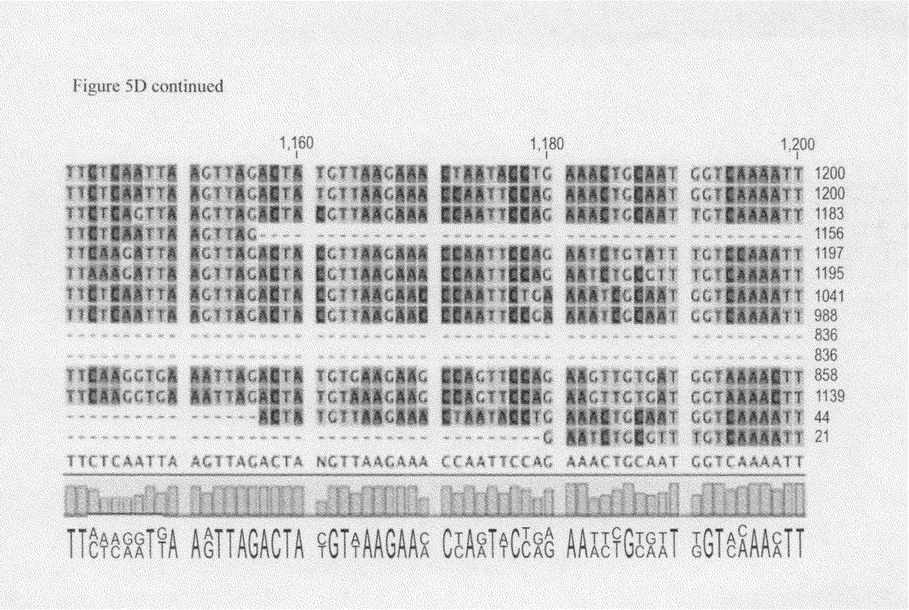

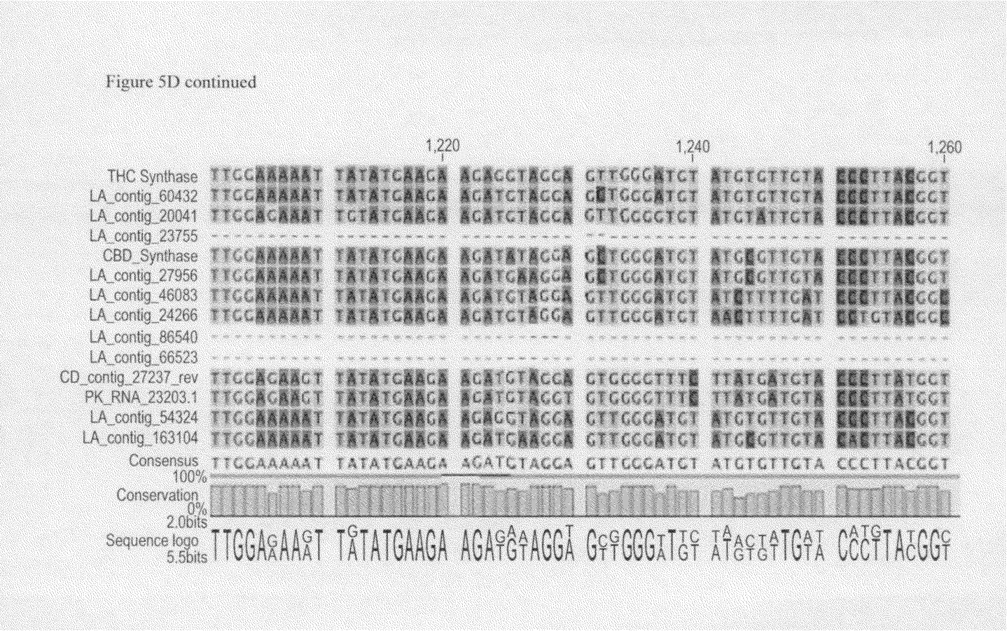

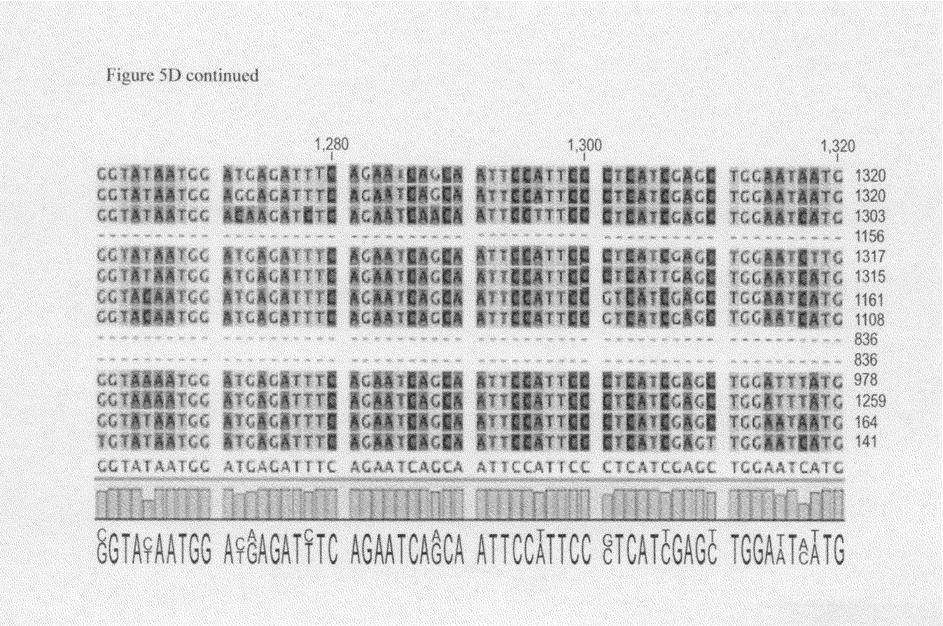

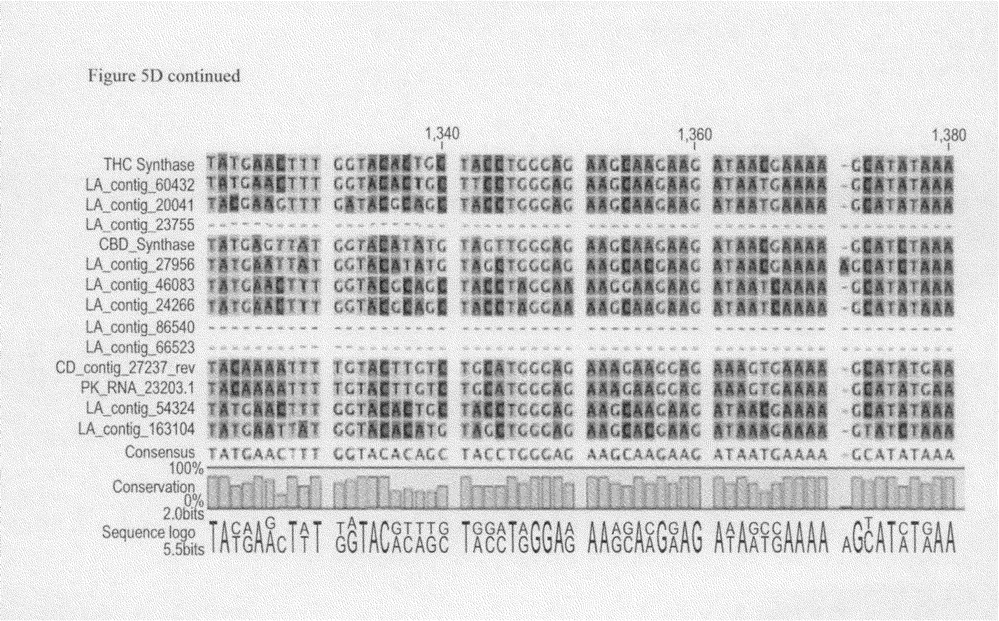

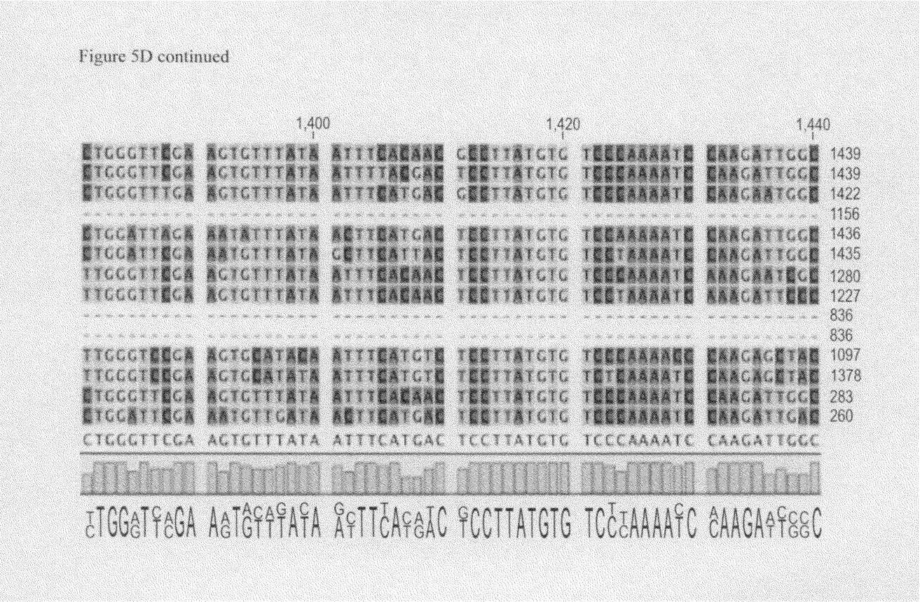

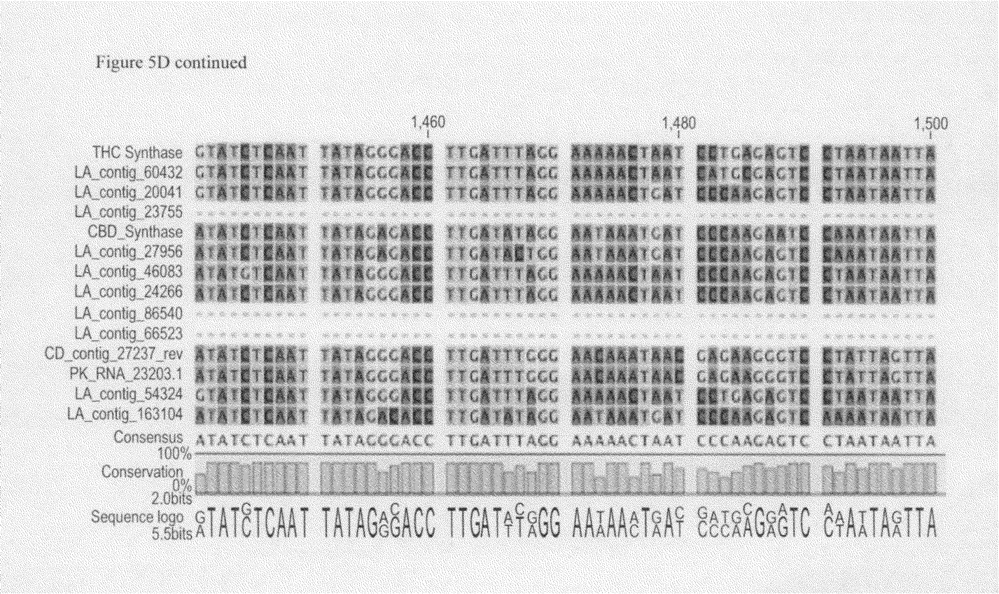

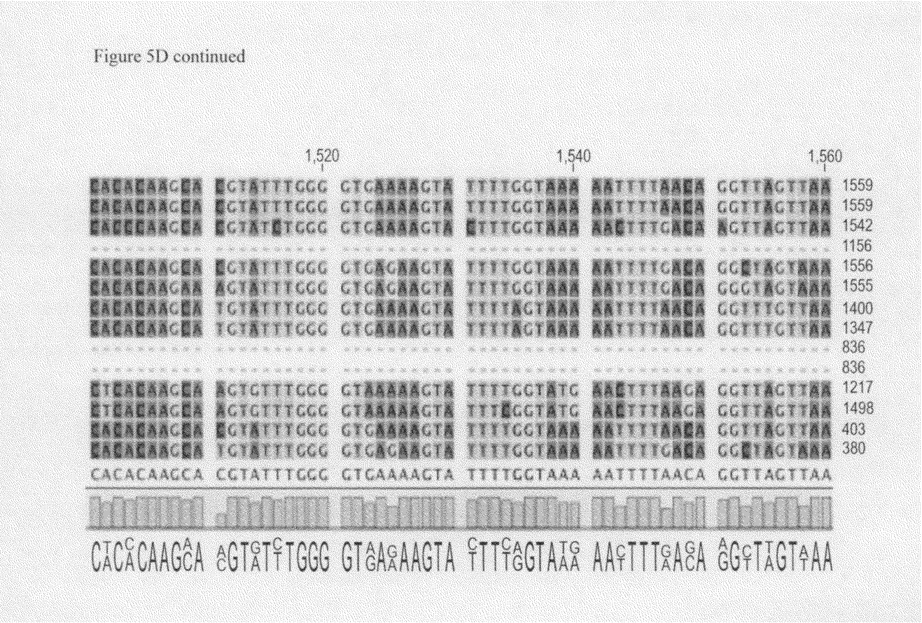

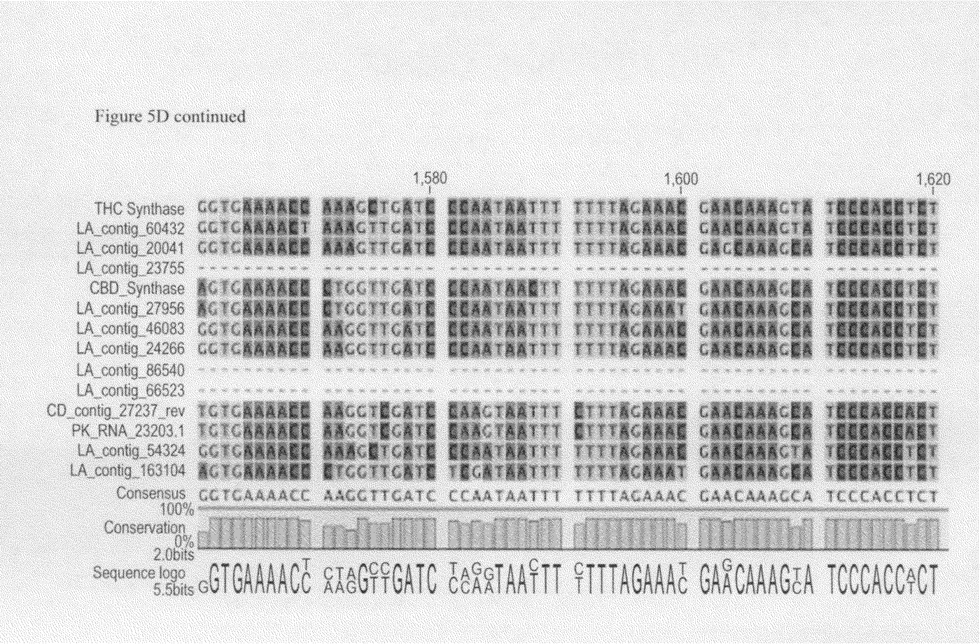

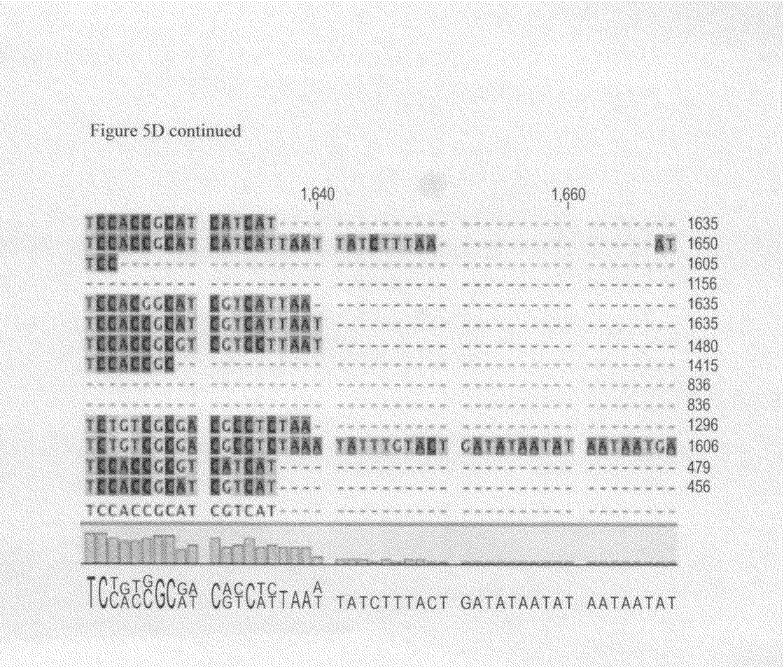

FIG. 4 shows these sequences as multiple sequence alignments and amino acid conservation plots show different 5′ and 3′ ends of the gene structures including internal amino acid substitutions (FIG. 5A to 5D). As a separate contig in the LAC Indica assembly, contig #34396 represents a 1650 bp ORF (coined MGC synthase-3 or MGC-s3) and is specifically expressed in the roots versus the flowers of PK Indica. The CSA assemblies of the Mexican cultivar from MPGR also confirm this expression pattern for this homologous contig csa_locus_{—}61504_iso_{—}1_len_{—}1623_ver_{—}2 across three Mexican cultivars. Furthermore, all cultivars (LAC Indica, CD Sativa, PK Indica, CSA), when expressed, maintained the FAD binding domain not seen active in the CBDA synthase alleles of LAC Indica (LAC CBDA Contig 27956 has a nonsense mutation 97 amino acids after the FAD binding site). The RSGGH and C176 amino acid sequences are critical for FAD crosslinking and exist in all versions of the peptide described herein.

### Synthase Gene Replication

Interestingly, many of the contigs containing THCA synthase genes have very high average genomic coverage due to cannabis LINE elements assembled at the edges of the contigs. In addition to LINE elements, the THCA synthase gene has an mPIF transposon signal of CWCTTAGWC at base 622. Others report the 3′ mPIF base has only 61% conservation, and thus cuts with star activity from its preferred recognition sequence of CWCTTAGWG. As with other mPIF transposons, a long inverted sequence is present 5′ to many of the assembled THCA synthase genes (FIG. 2B). If the THCA synthase gene recombines at base 626 (1635 bp full length) it would be expected to result in a truncated or significantly altered peptide, but the active site, including the FAD binding domain, would remain un-altered at base 165.

The increased coverage and polyploidy seen with the THCA and CBDA synthase genes in the Newbler-LAC assembly could be a result of a gene expansion generating a high diversity in the CBDA and THCA synthases. The unexplained diversity of cannabinoids discovered in the plant poses many open questions in regards to their modes of synthesis. These data provide additional context, providing at least four more synthase candidates to consider for the unknown genetic underpinnings of cannabichromene synthase or cannabichromene acid (CBCA). Others describe a 71 kDa CBCA synthase with a homodimer size of 136 kDa, and a 58-62 kDa range for synthases, with the remaining molecular weight being attributal to variable glycosylation. Further cloning and expression work is required to confirm catalytic activity of these putative genes. With the diversity of homolog or potentially paralog synthase sequences in the plant, one has to consider if the homodimers can, in fact, be heterodimers of similar synthase components, and if this combinatorial arrangement of peptides is responsible for the diversity of cannabinoid products in the plant. Such a model would favor rapid chemotype dominance seen with hyper expressive THCA synthase.

### Discussion

The findings of Harismendy and Lopez-Moreno suggest that polymorphism content in the human endocannabinoid pathway can better guide the selection or development of cultivars or pharmaceuticals with more favorable cannabinoid content. Independent isolation of cannabinoids has resulted in FDA approved drugs (THC or Marinol™), but studies have shown a 330% increase in efficacy with combined CBD and THC delivery resulting in the European approved Sativex™. Patients still report better outcomes from the whole plant extracts, re-enforcing the entourage effects described by Russo et al. and an interest in how each cultivar may vary in expression of active content. Towards this tailored end, GW Pharmaceuticals is now pursuing cultivars that express the varin or propyl side chain derivatives such as THCV as another therapeutic cannabinoid with less CB1 receptor affinity. In conclusion, complete dissection of the synthase gene repertoire and its precursors like AAE3 from van Bakel is imperative for predictive chemotyping of this valuable medicinal plant.

One of the challenges embarking on such studies is maintaining strong chain of custody of the plant matter to DNA, considering few countries have legal mechanisms to obtain plant material and legally sold cannabis has few quality and tracking standards to afford a properly designed genetic study. Material accessible through NIDA has been deemed less relevant as it fails to represent THC levels present in most strains used medicinally today.

As a result, the study described herein was aimed at sequencing one of the more popular C. sativa cultivars (“Chemdawg”) that has a controversial folklore over its origin to help underscore the value in a genetics based standard in the industry. Complimenting this was the sequence of a triple backcrossed C. Indica strain (“L.A. Confidential”) where legal entities are maintaining the seed line as clones (DNA Genetics, Netherlands). This sequence justifies further investigation into the genetics governing the cannabinoid and terpene expression. Future studies may consider a collaborative cross approach where stable inbred lines are carefully crossed to examine QTLs and alleles (Philip et al, 2011), and the various copies of THCA synthase can perhaps be better segregated and studied.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A nucleic acid comprising a nucleotide sequence that has about 82% to SEQ ID NO: 407,642, SEQ ID NO: 407,644, SEQ ID NO: 407,646 or SEQ ID NO:407 648 or a portion thereof that encodes a biologically active cannabinoid synthase, or a complement thereof.

2. The nucleic acid of claim 1 wherein the nucleic acid sequence comprises SEQ ID NO: 407,642, SEQ ID NO: 407,644, SEQ ID NO: 407,646 or SEQ ID NO: 407,648 or a portion thereof that encodes a biologically active cannabinoid synthase, or a complement thereof.

3. A polypeptide comprising an amino acid sequence that has about 67% identity to SEQ ID NO: 407,643, SEQ ID NO: 407,645, SEQ ID NO: 407,647 or SEQ ID NO: 407,649 or a biologically active portion thereof, such as a biologically active portion that functions as a cannabinoid synthase.

4. The polypeptide of claim 3 wherein the amino acid sequence comprises SEQ ID NO: 407,643, SEQ ID NO: 407,645, SEQ ID NO: 407,647 or SEQ ID NO: 407,649 or a biologically active portion thereof, such as a biologically active portion that functions as a cannabinoid synthase.

5. An antibody that specifically binds the polypeptide of claim 3.

6. A vector comprising the nucleic acid sequence of claim 1.

7. A cell comprising the vector of claim 6.

8. A method of producing a Cannabinoid synthase comprising maintaining the cell of claim 7 under conditions in which the Cannabinoid synthase gene is produced.

9. The method of claim 8 further comprising isolating the Cannabinoid synthase produced by the cell.

10. A Cannabinoid synthase gene produced by the method of claim 8.

11. A method of detecting a Cannabinoid in a sample comprising detecting the nucleic acid of claim 1 in the sample, wherein if the nucleic acid is detected, then a Cannabinoid is detected in the sample.

12. The method of claim 11 wherein the Cannabinoid is detected using a nucleic acid that hybridizes to all or a portion of the nucleic acid.

13. A method of detecting a Cannabinoid in a sample comprising detecting the polypeptide of claim 3, wherein if the polypeptide is detected, then a Cannabinoid is detected in the sample.

14. The method of claim 13 wherein the Cannabinoid is detected using a an antibody that binds to the polypeptide.

15. A method of detecting one or more cannabinoid genes in a Cannabis plant comprising:
a) contacting all or a portion of a genomic sequence of the Cannabis plant with one or more primers that are complementary to SEQ ID NO: 407,642, SEQ ID NO: 407,644, SEQ ID NO: 407,646, SEQ ID NO: 407,648 or a combination thereof, thereby producing a reaction mixture;
b) maintaining the reaction mixture under conditions in which one or more sequences in the genomic sequence of the Cannabis plant that are complementary to one or more of the primers hybridize to the one or more primers;
c) amplifying the one or more sequences that hybridize to the one or more primers, thereby producing one or more amplicons; and
d) determining all or a portion of the sequence of the one or more amplicons, thereby detecting one or more cannabinoid genes in the Cannabis plant.

16. The method of claim 15 further comprising quantifying the one or more Cannbinoid genes.

17. The method of claim 16 wherein the one or more Cannabinoid genes are quantified by labeling the amplicons, detecting the labeled amplicons in real time and quantifying the labeling amplicons as the amplicons are generated.

18. The method of claim 17 further comprising contacting the reaction mixture with reverse transcriptase to measure Cannabinoid messenger ribonucleic acid (mRNA).

19. The method of claims 15 further comprising detecting whether fungal nucleic acid, bacterial nucleic acid, or a combination thereof is present.

20. The method of claim 19 wherein if fungal nucleic acid, bacterial nucleic acid, or a combination thereof is present, then the method further comprises quantifying the fungal nucleic acid, bacterial nucleic acid, or a combination thereof.

21. The method of claim 20 further comprising comparing the quantified fungal nucleic acid, bacterial nucleic acid, or a combination thereof to the quantified cannabinoid nucleic acid.
